(19) United States
(12) Patent Application Publication
(10) Pub. No.: US 2005/0171233 A1
(43) Pub. Date: Aug. 4, 2005

(54) Two-component dental material crosslinking by addition, by way of a hydrosilylation reaction, having rigid and/or voluminous groups as well as great flexural strength

(75) Inventors: Alexander Bublewitz, Herborn (DE); Jens-Peter Reber, Meinerzhagen (DE); Ulrich Nagel, Tubingen (DE)

Correspondence Address: WILLIAM COLLARD;COLLARD & ROE, P.C., 1077 NORTHERN BOULEVARD, ROSLYN, NY, 11576, US

(73) Assignee: Kettenbach GmbH & Co. KG

(21) Appl. No.: 11/045,920

(22) Filed: Jan. 28, 2005

(30) Foreign Application Priority Data
Feb. 3, 2004 (DE) 10 2004 005 562.9

(51) Int. Cl. A61K 6/08

(52) U.S. Cl. 523/116

(57) ABSTRACT

A two-component dental material addition-crosslinking by way of hydrosilylation contains (a) one or more compounds having vinyl groups in the molecule, (b) at least one organohydrogen silicone compound, and (c) at least one catalyst. The at least one compound (a) and/or the at least one compound (b) includes as the first structural unit, at least one voluminous and/or rigid group, and as the second structural unit, at least two alkenyl-functional or at least two hydrogen-functional silyl units. The second structural unit is bound to the first structural unit (i) directly, (ii) by way of an oxygen atom, (iii) by way of a spacer group, or (iv) by way of a spacer group according to (iii), which is bound to the first structural unit by way of an oxygen atom.

### CROSS REFERENCE TO RELATED APPLICATIONS

Applicants claim priority under 35 U.S.C. §119 of German Application No. 10 2004 005 562.9 filed Feb. 3, 2004.

### BACKGROUND OF THE INVENTION

1. Field of the Invention

The present invention relates to a two-component, dental material crosslinking by addition, by way of hydrosilylation, containing one or more compounds having vinyl groups in the molecule, at least one organohydrogen silicon compound, and at least one catalyst. In addition, the present invention relates to the use of these addition-crosslinking two-component dental materials.

2. The Prior Art

In the case of various measures in the sector of dental medicine and dental technology, for example for taking tooth impressions, bite registration, fixation, positioning, keying, restoration, transfer of brackets in gnathic orthopedics, and repositioning of fillings for grinding the occlusion on the metal, materials are required that make a transition from a viscous state into a particularly hard state. Traditionally, addition-crosslinking two-component silicone materials are used for this purpose. One of the two components of this composition contains polysiloxanes capable of crosslinking, particularly organopolysiloxanes having at least two vinyl groups per molecule, as well as a hydrosilylation catalyst required for the crosslinking reaction, typically a platinum compound. The other component includes at least one hydrogen polysiloxane, usually an organohydrogen polysiloxane, as the crosslinking agent. In addition, one or both components can contain reinforcing fillers, non-reinforcing fillers and/or additional additives and processing aids, such as dyes or the like. To adjust the curing time, which determines the working time, inhibitors, for example organopolysiloxanes that contain short-chain vinyl groups, particularly organodisiloxanes, benzotriazol, or ethinyl cyclohexanol, are added to the silicone materials. The mechanical properties, such as hardness and modulus of elasticity, are particularly determined by means of the chain length of the organopolysiloxanes used, as well as the type and amount of fillers.

Addition-crosslinking masses on a polysiloxane basis are known from EP 0 522 341 A1. These masses contain not only organopolysiloxanes having two or more vinyl groups in the molecule and a viscosity between 100 and 200,000 mPa.s, organohydrogen polysiloxanes as crosslinking agents, a catalyst, and dyes, but also compounds of highly disperse active fillers in silicone oil and short-chain organopolysiloxanes having two or more vinyl groups in the molecule. The short-chain organopolysiloxanes correspond to the general formula
CH₂═CH—R₂SiO—(SiR₂O)ₙ—SiR₂—CH═CH₂
where R stands for the same or different hydrocarbons, free of aliphatic multiple bonds, univalent, if necessary substituted, and n stands for a whole number between 10 and 20. However, the silicone materials disclosed in EP 0 522 341 A1 merely have a Shore A hardness of a maximum of 78, corresponding to a Shore D hardness of a maximum of 19, and a modulus of elasticity, in the tensile test, of a maximum of 9 MPa. These mechanical properties are insufficient for most uses in dental medicine and dental technology, particularly for bite registration.

In EP 0 894 117 B1, addition-crosslinking two-component silicone materials are disclosed. These materials contain organopolysiloxanes having two vinyl groups in the molecule, organohydrogen polysiloxanes having two or more SiH groups and an SiH content of 1 to 15 mmol/g as a crosslinking agent, a catalyst, reinforcing fillers, as well as non-reinforcing fillers, whereby the organopolysiloxane having two vinyl groups in the molecule has a viscosity between 21 and 99 mPa.s, corresponding to a chain length of 21 to 69. These cured masses have a Shore D hardness of at least 35 and a modulus of elasticity of greater than 20 MPa (measured according to DIN 53457 or 53455), which values are quite good for most uses in dental medicine and dental technology. Nevertheless, materials having greater mechanical strength and lower elasticity are desirable for these indications.

### SUMMARY OF THE INVENTION

Therefore, the present invention aims at providing a dental material having a greater Shore D hardness or a higher modulus of elasticity, or both, as compared with the known masses. This dental material should also be at least comparable to the known masses with regard to its other applications and technical properties, particularly with regard to its degree of shrinkage and working time.

According to one aspect of the invention, this object is accomplished by means of a two-component dental material crosslinking by addition, by way of hydrosilylation, containing
(a) one or more compounds having vinyl groups in the molecule,
(b) at least one organohydrogen silicon compound, and
(c) at least one catalyst,
wherein the at least one compound (a) and/or the at least one compound (b) includes:
as the first structural unit, at least one voluminous and/or rigid group, preferably one selected from the group consisting of tertiary alkyl, quaternary alkyl, cycloalkyl, cycloalkenyl, aryl, aralkyl, alkylaryl groups, halogen-substituted tertiary alkyl groups, halogen-substituted quaternary alkyl groups, halogenated aryl groups, halogenated aralkyl groups, halogenated alkylaryl groups, particularly preferably aromatic and non-aromatic mono, bis, oligo, polycyclic groups, bisphenol A, bisphenol B, bisphenol F groups, 1,1,1-tris(4-hydroxyphenyl)alkane groups, norbornane groups, adamantane groups, and pentaerythrite groups, as well as
as the second structural unit, at least two alkenyl-functional or at least two, preferably at least three hydrogen-functional silyl units having the general formula I 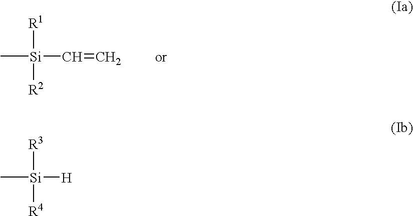
   where R¹, R², independent of one another, are selected from the group consisting of alkyl groups, alkenyl groups, aryl groups, aralkyl groups, alkylaryl groups, halogenated alkyl groups, halogenated aryl groups, halogenated aralkyl groups, halogenated alkylaryl groups, cyanoalkyl groups, siloxy groups, cycloalkyl groups, and cycloalkenyl groups, wherein alkyl groups are particularly preferred and methyl groups are very particularly preferred, and R³, R⁴, independent of one another, are H or R¹, wherein alkyl groups are particularly preferred and methyl groups are very particularly preferred,
wherein the second structural unit is bound to the first structural unit
(i) directly,
(ii) by way of an oxygen atom,
(iii) by way of a spacer group, or
(iv) by way of a spacer group according to (iii), which is bound to the first structural unit by way of an oxygen atom.

### DETAILED DESCRIPTION OF PREFERRED EMBODIMENTS

The present invention is based on the surprising recognition, which was completely unexpected for a person skilled in the art, that compositions containing at least one compound (a) and/or at least one compound (b) having at least one voluminous and/or rigid group, along with the other compounds (a), (b), and (c), in each instance, cure to form masses having excellent mechanical properties, particularly outstanding strength and a high modulus of elasticity, and are furthermore excellently suited for uses in dental medicine and dental technology.

In particular, these materials cure in a time that is sufficiently short for uses in dental medicine and dental technology. The excellent mechanical properties and other application technology related properties of the compositions according to the invention are attributable to the voluminous and/or rigid group of the compound (a) and/or to the voluminous or rigid group of the compound (b).

Preferably, the individual components are selected so that the Shore D hardness of the dental materials according to the invention is greater than 35 in the fully vulcanized state (24 hours after curing at room temperature), particularly preferably greater than 50, and very particularly preferably greater than 69, and/or the flexural strength is at least 8 MPa, particularly preferably at least 15 MPa, and very particularly preferably at least 19 MPa, and/or the modulus of elasticity in the bending test (measured according to ISO 10477) is at least 300 MPa, preferably at least 600 MPa, and very particularly preferably at least 900 MPa. At the same time, the setting time is preferably less than 10 minutes, and particularly preferably less than 5 minutes.

Fundamentally, all rigid and/or voluminous groups are suitable as the first structural unit, i.e. as the voluminous and/or rigid group. Rigid groups in the sense of the present invention are understood to be groups having a defined, inflexible three-dimensional structure. Voluminous group in the sense of the present invention refers to those groups that because of their size, taking into consideration the reciprocal orientation of the vinyl groups in the molecule, guarantee that the distance between the at least two vinyl groups per molecule of the compound (a), in each instance, is at least 0.3, particularly at least 0.45, at least 0.5, at least 0.55, at least 0.6, at least 0.7, at least 0.8, or at least 0.9 nm, respectively.

This feature leads to the result that with a decreasing angle of the vinyl groups from one another in the molecule, the first structural unit must have an all the greater radius of an imaginary sphere around the groups, in order to achieve the required aforementioned distances between the vinyl groups relative to one another. The present invention is based on the surprising recognition that at a distance of the vinyl groups above the aforementioned limits, an undesirable chelate effect due to approach of two vinyl groups of a molecule to a platinum atom can be reliably prevented. The aforementioned structural parameters apply analogously for the compound (b).

According to the invention, the first structural unit, i.e. the voluminous and/or rigid group, is one that is selected from the group consisting of tertiary alkyl, quaternary alkyl, cycloalkyl, cycloalkenyl, aryl, aralkyl, alkylaryl groups, halogen-substituted tertiary alkyl groups, halogen-substituted quaternary alkyl groups, halogenated aryl groups, halogenated aralkyl groups, halogenated alkylaryl groups, particularly preferably aromatic and non-aromatic mono, bis, oligo, polycyclic groups, bisphenol A, bisphenol B, bisphenol F groups, 1,1,1-tris(4-hydroxyphenyl)alkane groups, norbornane groups, adamantane groups, and pentaerythrite groups.

The following structures, in particular, have proven themselves to be particularly suitable as the first structural unit: 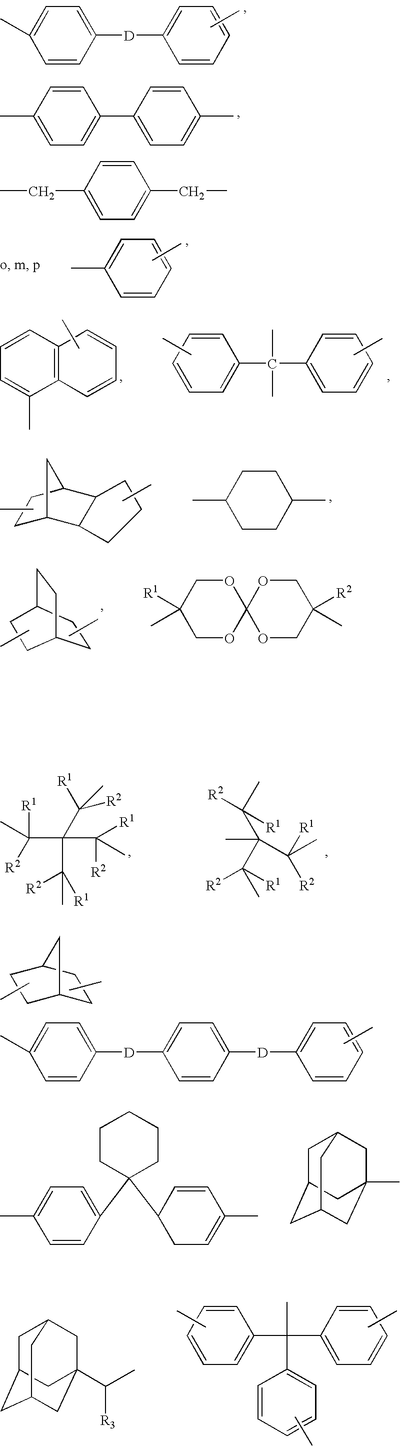
where R¹, R², R³ are as defined in the formulas I or hydrogen, D=CR³R⁴ with R³ and R⁴ as defined above, particularly D equals CH₂, C(CH₃)₂, CMePh with Me═CH₃ and Ph═C₆H₅ or CPh₂. 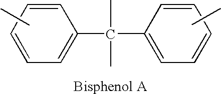
bisphenol A derivatives, and trisphenol compounds 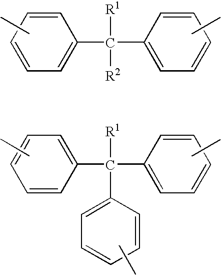
with R¹, R² as defined above are very particularly preferred as the first structural unit.

As the second structural unit, i.e. as the hydrogen-functional and/or alkenyl-functional silyl unit, those having the general formulas Ia or Ib have particularly proven themselves, in which the radicals R¹, R² and R³, R⁴, respectively, independent of one another, are selected from the group consisting of alkyl groups, alkenyl groups, aryl groups, aralkyl groups, alkylaryl groups, halogenated alkyl groups, halogenated aryl groups, halogenated aralkyl groups, halogenated alkylaryl groups, cyanoalkyl groups, siloxy groups, cycloalkyl groups, and cycloalkenyl groups, whereby R³, R⁴ can also be hydrogen radicals. Particularly preferably, the radicals R¹, R², independent of one another, are alkyl groups, aryl groups, aralkyl groups, or alkylaryl groups, very particularly preferably methyl or phenyl groups, and the radicals R³, R⁴, independent of one another, are hydrogen, alkyl groups, aryl groups, aralkyl groups, or alkylaryl groups, very particularly preferably methyl or phenyl groups.

According to a first embodiment of the present invention, the addition-crosslinking two-component dental material contains at least one compound (a), (b), and (c). The at least one compound (a) and/or the at least one compound (b) is made up of at least one of the stated first as well as at least one of the stated second structural units, wherein the at least one first structural unit is directly bound to the at least one second structural unit, and therefore falls under one of the general formulas II
H₂C═CH—SiR¹R²-Eₙ₁-SiR¹R²—CH═CH₂ (IIa)
and/or
H—SiR³R⁴-Eₙ₁-SiR³R⁴—H (IIb),
wherein the radicals R¹, R², R³, R⁴ are as defined in formula I, E refers to the first structural unit, and n₁ is a whole number ≧1, preferably 1.

Preferably, the compound (a) falling under the formula IIa, or the compound (b) falling under the formula IIb, has only one rigid and/or voluminous group (n₁=1), flanked by two structural units having the general formula Ia or Ib, whereby the group E, however, can certainly also be substituted with two to six groups having the general formula I.

Good results are also achieved according to a second embodiment of the present invention, if the at least one compound (a) and/or the at least one compound (b) is made up of at least one of the stated first as well as at least one of the stated second structural units, wherein the at least one first structural unit is bound to the at least one second structural unit by way of an oxygen atom, and thereby falls under the general formulas III
H₂C═CH—SiR¹R²—O-Eₙ₁-O—SiR¹R²—H—CH₂ (IIIa),
and/or
H—SiR³R⁴—O-Eₙ₁-O—SiR³R⁴—H (IIIb)
wherein the radicals R¹, R², R³, R⁴ and E as well as n₁ are defined as above.

In contrast to the compounds according to formulas II, these substances have Si—O—C bonds, which are considered to be sensitive to hydrolysis, according to the state of the art, and therefore unsuitable for dental materials, because of the hydrolysis caused by the saliva in the patient's mouth. Surprisingly, it was now found, within the scope of the present invention, that Si—O—C bonds in compounds having the general formulas III are by no means generally sensitive to hydrolysis, but rather can be stable against hydrolysis. These compounds are stable against hydrolysis, according to the present invention, if the carbon atom of the Si—O—C bond is substituted with at least one alkyl, aryl, or aralkyl group, which can have additional substituents.

Furthermore, even compounds (a) having Si—O—C bonds, that are sensitive to hydrolysis, in which the carbon atom is substituted only with hydrogen, can be used, if these compounds are mixed with organohydrogen silicon compounds (b) that do not contain Si—O—C, and cured, as well as vice versa, compounds (b) that are sensitive to hydrolysis, having Si—O—C bonds in which the carbon atom is substituted only with hydrogen, can be used if these compounds are mixed with compounds (a) that do not contain vinyl groups having Si—O—C bonds, and cured. The vulcanizates that are obtained are stable against hydrolysis after storage in water, and this stability is obviously attributable to a shielding effect of the hydrophobic silicon groups.

According to a third embodiment of the present invention, the at least one compound (a) and/or the at least one compound (b) of the dental material according to the invention is made up of at least one of the stated first as well as at least one of the stated second structural units, wherein the at least one first structural unit is bound to the at least one second structural unit by way of a spacer A, and thereby falls under the general formulas IV
H₂C═CH—SiR¹R²-A¹ₙ₂-Eₙ₁-A²ₙ₃-SiR¹R²—CH═CH₂ (IVa)
and/or
H—Si R³R⁴-A¹ₙ₂-Eₙ₁-A²ₙ₃-SiR³R⁴—H (IVb),
wherein the radicals R¹, R², R³, R⁴ and E as well as n₁ are defined as above, n₂, n₃ are the same or different, and are whole numbers ≧1, preferably 1, in each instance, and A¹, A², independent of one another, is a spacer.

All of the structures known to a person skilled in the art to be suitable for this purpose are suitable as spacer A. In particular, alkyl, alkoxy, alkenyl, alkylenoxy, cycloalkyl, cycloalkoxy, cycloalkenyl, cycloalkenyloxy, aryl, aralkyl, alkylaryl, aroxy, aralkoxy, alkylaroxy, cyanoalkyl, cyanoalkoxy, halogen-substituted alkyl groups, halogenated aryl groups, halogenated aralkyl groups, halogenated alkylaryl groups, halogen-substituted alkoxy groups, halogenated aralkoxy groups, and halogenated alkylaroxy groups are suitable. Particularly preferred as spacers are mono, bis, oligo, polyether structural units or polydialkyl siloxane structural units ([—O—SiR¹R²]ₙ, where R¹, R² are as defined above), with a repetition unit n of the ether or the siloxane of n=1 to 20, whereby the ether structural unit very particularly preferably falls under one of the general formulas V
—(CR⁵₂—CR⁵₂—O)ₙ₄ (Va)
and/or
—(CR⁵₂—CR⁵₂—CR⁵₂—CR⁵₂—O)ₙ₄ (Vb),
wherein the radicals R⁵, independent of one another, are H, alkyl, aryl, aralkyl, alkylaryl, particularly preferably H or a C₁-C₅ alkyl radical, and n₄ is a whole number ≧1, preferably between 1 and 20.

The following groups have proven themselves to be highly preferably suitable as spacers, within the scope of the present invention:
—OCH₂CH₂—, —O(CH₂)₄—, —O—CR¹R²—CR¹R²— as well as particularly preferably —CH₂CH₂CH₂(OCH₂CH₂)₄—, —CH₂—CH₂—CH₂—, —O—C(CH₃)₂—CH₂—, —O—CH(CH₃)—CH₂—, —O—CH(C₂H₅)—CH₂—, —O—CH(C₄H₉)—CH₂— and —O—CH(C₁₀H₂₁)—CH₂—.

Good results are also achieved according to a fourth embodiment of the present invention, if the at least one compound (a) and/or the at least one compound (b) is made up of at least one of the stated first as well as at least one of the stated second structural units, wherein the at least one second structural unit is bound to the first structural unit by way of a spacer according to the third embodiment, which is bound to the at least one first structural unit by way of an oxygen atom.

Preferably, only those having a voluminous and/or rigid group, in other words those in which n₁ is equal to 1, in the general aforementioned formulas II to IV, are used as the compound (a) and/or compound (b).

Preferably, the at least one compound (a) has an Si vinyl content of 0.5 to 10 mmol/g, particularly preferably from 1 to 10 mmol/g, and very particularly preferably from 2 to 10 mmol/g.

Particularly good application technology properties with regard to mechanics and optical properties are demonstrated by the dental materials according to the invention that contain one of the following substances as compound (a), wherein the radicals R¹, R², unless otherwise noted in the formulas below, have the meaning given in the legend for formula Ia: 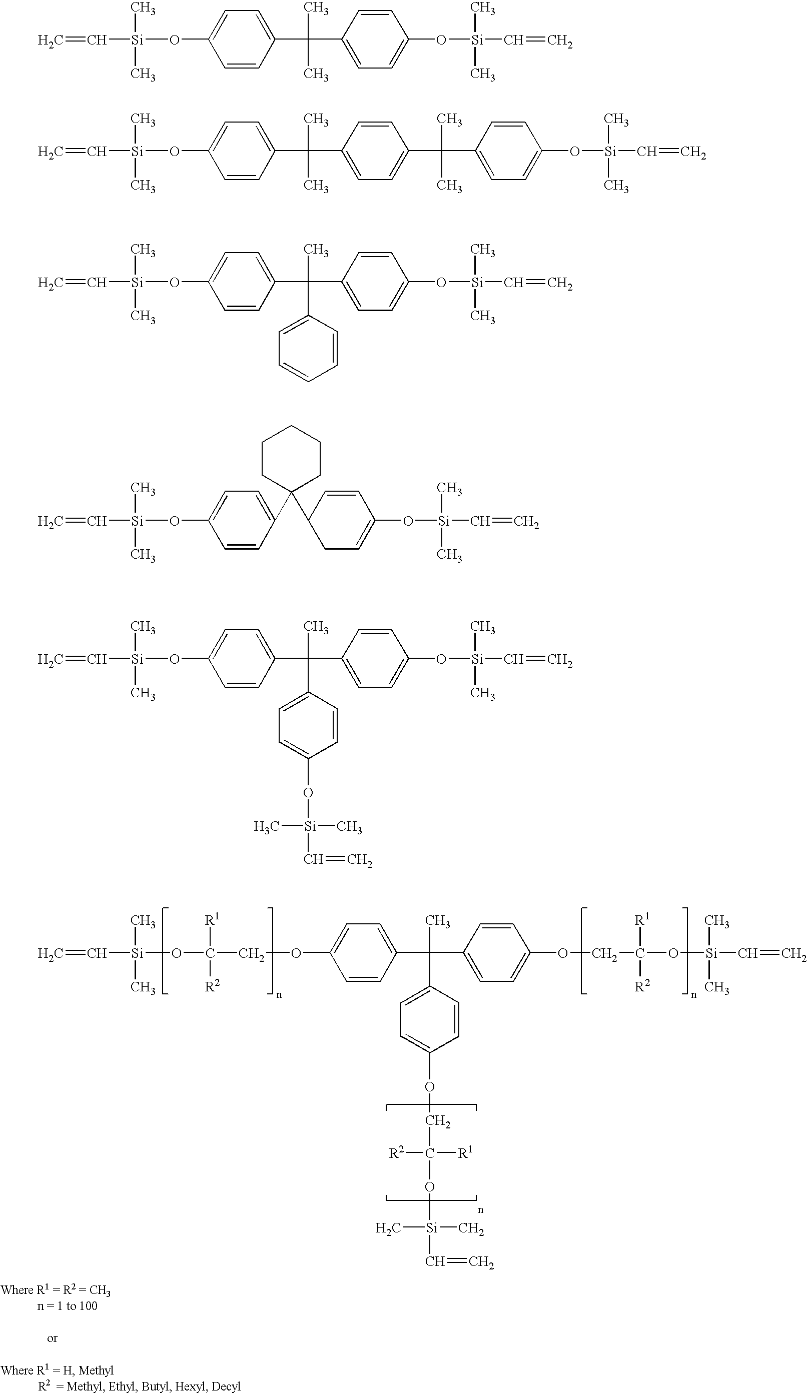 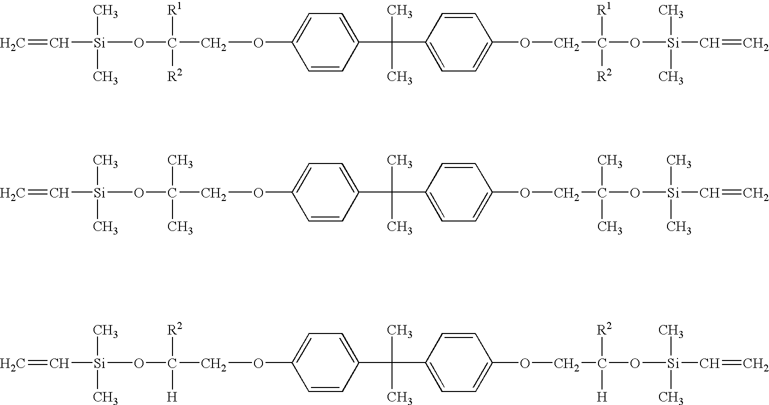
where R²=Methyl, Ethyl, Butyl, Hexyl, Decyl 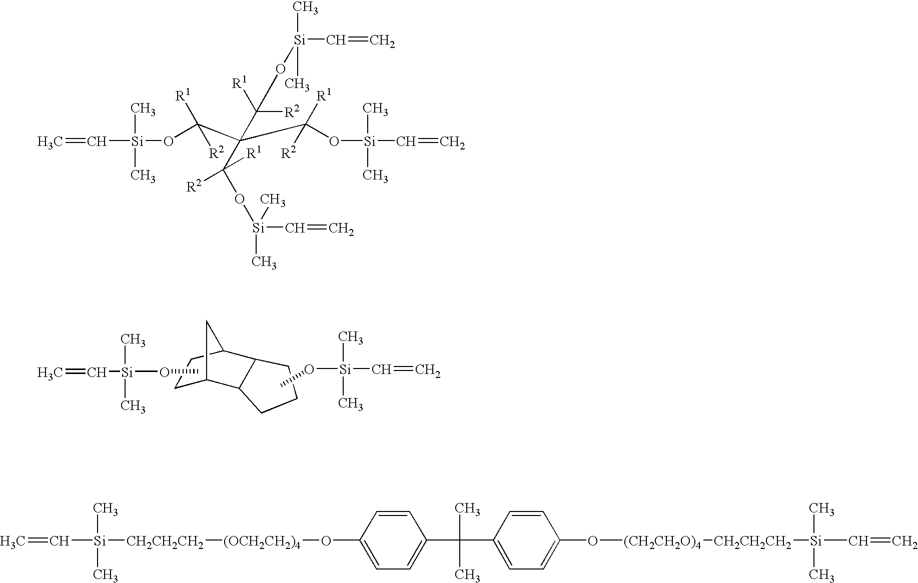
where n is between 0 and 100

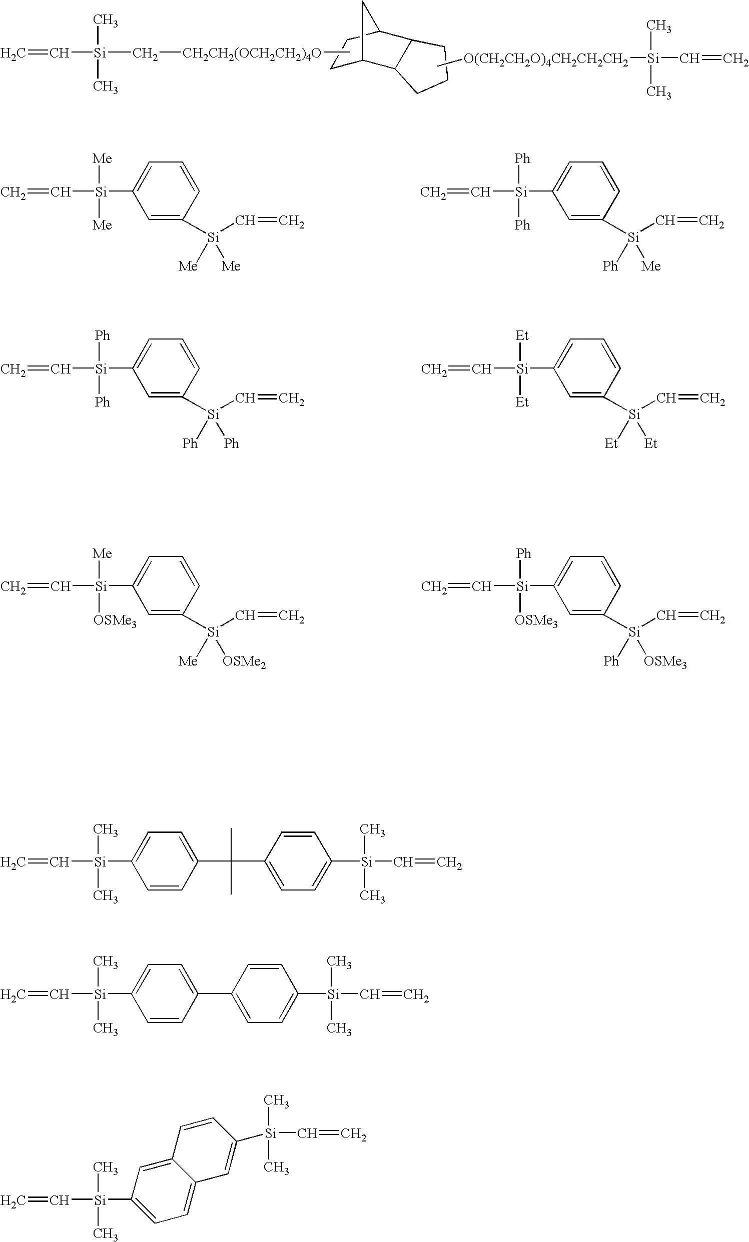

According to the invention, the dental material contains at least one compound (a) having the stated two structural units, or at least one compound (b). It is particularly preferred for the dental material to also contain at least one corresponding compound (a) and (b). In the case that only one compound (a) having the stated two structural units is contained in them, the dental materials according to the invention can also have one or more organopolysiloxanes known according to the state of the art, having at least two vinyl groups per molecule and a viscosity (measured at 20° C.) between 21 and 350,000 mPa.s (components a₂) added to them, whereby the content of these compounds is preferably 0 to 40 wt.-%, particularly preferably 0 to 30 wt.-%, and very particularly preferably 0 to 20 wt.-%.

If the dental material according to the invention has only compound (a) having at least one rigid and/or voluminous group in the sense of the present invention, but no corresponding compound (b), all of the organohydrogen polysiloxanes known to a person skilled in the art, for this purpose, can be used as crosslinking agents. In particular, polyalkyl, polyaryl, polyaralkyl, polyhalogen alkyl, polyhalogen aryl, and polyhalogen aralkyl siloxanes, which have at least two, preferably at least three hydrogen atoms bound to silicon atoms in the molecule, have proven themselves to be suitable.

If the dental material according to the invention contains at least one compound (b) having a rigid and/or voluminous group in the sense of the present invention, organohydrogen polysiloxanes known from the state of the art (component b₂) can also be added to this list of crosslinking agents, preferably those having at least two Si—H groups per molecule and an Si—H content between 0.1 and 15 mmol/g, particularly preferably between 4 and 14 mmol/g, and very particularly preferably between 5 and 13 mmol/g, as well as a viscosity (at 20° C.) of 5 to 2,000 mPa.s. In this case, the total of organohydrogen polysiloxanes having at least one rigid and/or voluminous group in the sense of the present invention and of organohydrogen polysiloxanes according to the state of the art is at least 1 weight percent.

Preferably,
linear organohydrogen polysiloxanes having at least two Si—H groups having the general formula VI 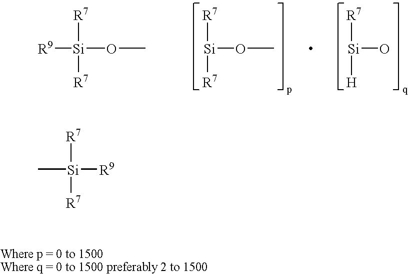
   where R⁷=alkyl (e.g. methyl, ethyl, isopropyl), aryl (e.g. phenyl, naphthyl, tolyl, xylyl), aralkyl (benzyl, phenylethyl) and halogen-substituted alkyl and aryl groups (e.g. 3,3,3-trifluoropropyl, chlorophenyl, difluorophanyl), cyanalkyl, cycloalkyl, and cycloalkenyl. Preferably, R=methyl,
where R⁹=R⁷ and/or H
with the proviso that at least 2 Si atoms of the formula VI bear an H atom;
oraganohydrogen polysiloxanes having T structural units having the general formula VII 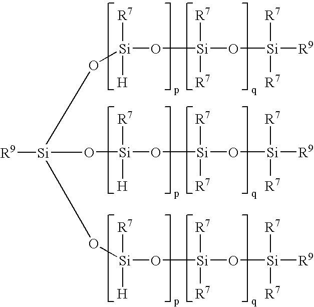
   wherein the radicals have the meaning given in the formula VI
   and/or
the formula VIII:
   [SiO_{4/2}][SiO_{1/2}]R¹R²H]ₘ where m=0.9 to 4, preferably m=1 to 4
   and/or
QM resins containing Si—H, made up of (R⁹)₃SiO_{1/2}, (R⁷)₂(H)SiO_{1/2} and SiO_{4/2} units, wherein the trifunctional (R⁹)₁SiO_{3/2} can also be present as T units, and the bifunctional R⁷R⁹SiO_{2/2} can also be present as D units, wherein the radicals R⁷ and R⁹ have the meaning as indicated previously,
   are used as organohydrogen silicon compounds.

Particularly preferably, the at least one organohydrogen silicon compound (b) has aromatic mono, bis, oligo or polycyclus and/or a non-aromatic mono, bis, oligo or polycyclus as the first structural unit, i.e. as the rigid and/or voluminous group, as well as those in which the radicals R³, R⁴, independent of one another, are hydrogen, alkyl, aryl, aralkyl, or alkylaryl groups, very particularly preferably methyl or phenyl groups as the hydrogen-functional silyl groups.

Examples of suitable organohydrogen silicon compounds—wherein the radicals R¹, R², unless otherwise noted in the following formulas, have the meanings indicated above, are: 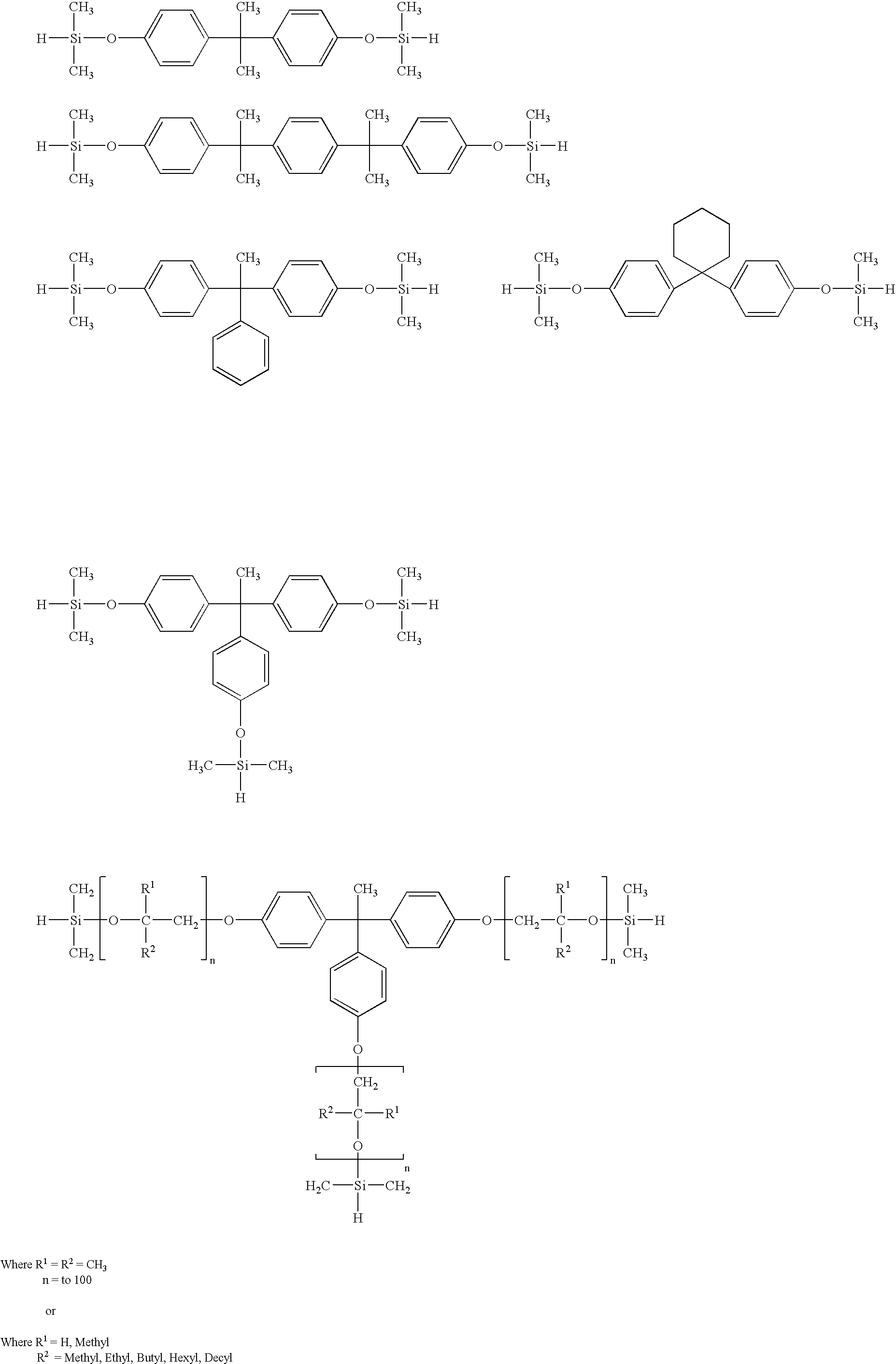 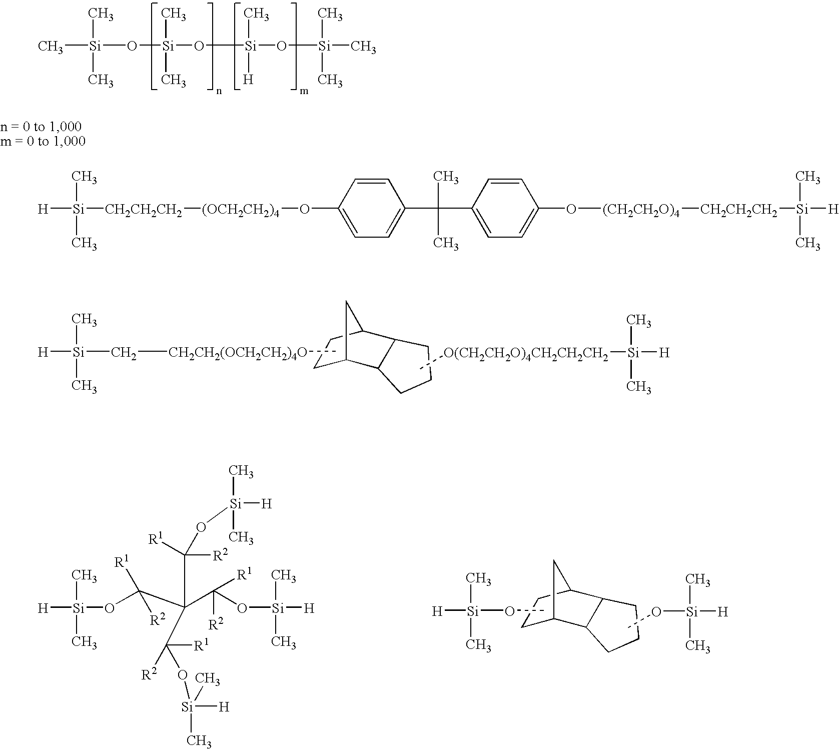
as well as Silopren® U430, U730, U830, and particularly preferably U930 from Bayer.

In order to minimize or completely prevent hydrogen molecules from gassing out of the crosslinking agent molecules, organohydrogen silicon compounds that have no Si—H bonds at adjacent silicon atoms, in each instance, but rather bonds that contain the following structural unit, for example, are particularly preferably used: 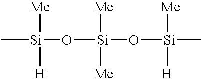
where Me=CH₃.

Furthermore, organohydrogen silicon compounds having a T or Q structure are particularly preferably used, since they have a particularly high Si—H content.

Preferably, the organohydrogen silicon compound (b) has an SiH content of 0.1 to 15 mmol/g, particularly preferably of 4 to 15 mmol/g, and very particularly preferably of 7 to 15 mmol/g, as well as a viscosity of 1 to 10,000 mPa.s, particularly preferably 5 to 2,000 mPa.s (measured at 20° C using a Haake viscosimeter).

All of the substances known to a person skilled in the art for this purpose can be used as catalysts (component c) for the hydrosilylation, particularly salts, complexes, and forms of the transition metals of the 8^{th} secondary group that are present in colloidal form, preferably of the metals platinum, palladium, and rhodium. Preferably, the compositions according to the invention contain platinum complexes as catalysts, which are produced, for example, from hexachloroplatinum acid or from corresponding platinum salts, and particularly preferably platinum divinyl tetramethyl disiloxane.

Aside from at least one compound (a) and at least one compound (b), wherein at least one compound (a) and/or at least one compound (b) has a rigid and/or voluminous group in the sense of the present invention, as well as at least one catalyst (c) and, if necessary, one or more compounds (a₂), (b₂), the two-component dental materials according to the invention can additionally contain one or more substances from one or more of the following groups, in each instance:
(d) reinforcing fillers,
(e) non-reinforcing fillers,
(f) dyes,
(g) moisture binders,
(h) inhibitors,
(i) QM resins that contain vinyl groups, Si—H groups, and/or Si—OR groups (where R═H or alkyl),
(j) compounds of organopolysiloxane and reinforcing fillers,
(k) surfactants, emulsifiers and/or stabilizers,
(l) radio-opaque substances,
(m) substances that absorb or adsorb H₂, and substances that eliminate or reduce H₂ development, as well as
(n) other aids and additives, such as rheology aids, X-ray contrast compounds, antimicrobial substances, astringent substances, or the like.

Highly disperse, active fillers having a BET surface of at least 50 m²/g (see monograph series Dyes Degussa Kieselsauren [Dyes Degussa Silicic Acids], Number 12, page 5, as well as Number 13, page 3) are particularly suitable as component (d). For example, titanium dioxide, aluminium oxide, zinc oxide, zirconium oxide and, particularly preferably, wet-precipitated or pyrogenically obtained silicic acid are suitable as component (d). The substances named can be present in hydrophilic or hydrophobized form. Furthermore, nanoparticles and fiber-form or lamella-form fillers can be used as reinforcing fillers, whereby mineral fiber-form fillers, such as woolastonite, and synthetic fiber-form fillers, for example glass fibers, ceramic fibers, or plastic fibers, are preferred. Nanoparticle in the context of the present invention refers to specially produced inorganic or organic powders whose average grain size is less than 100 nanometers.

Furthermore, the compositions according to the invention can contain one or more non-reinforcing fillers. Preferably those fillers have a BET surface of less than 50 m²/g. Particularly preferably, the non-reinforcing fillers used have an average grain size of ≧0.1 μm (Ullmann's Encyclopädie der Technischen Chemie [Encyclopedia of Technical Chemistry], Volume 21, page 523). Particularly preferably, the non-reinforcing fillers are selected from the group consisting of metal oxides, metal hydroxides, metal oxide hydroxides, mixed oxides, and mixed hydroxides. Fillers such as calcium carbonate, silica gel, diatomaceous earth, talcum, and fillers on a plastic basis, for example polyalkyl (meth)acrylate, polycarbonate, polyvinyl chloride, silicone resin powder, powder on the basis of fluoro-organic compounds, as well as organic and inorganic hollow beads, solid beads, and fibers can be used. Furthermore, solid or hollow plastic particles, for example also in spherical shape, on the surface of which inorganic filler particles are embedded, can be used. Silicon dioxide, particularly in the form of quartz and its crystalline modifications, translucent fused quartz as well as quartz powder, cristobalite, dental glass, dental ceramic, aluminium oxide, calcium oxide, and aluminium hydroxide are preferred.

The fillers named under (d) and (e) can also be present in surface-treated (coated) form. The surface treatment or coating can be made, for example, with silanes and fatty acids, which can have functional groups (e.g. vinyl, Si-vinyl, allyl, —SiH, acryl and methacryl). The fillers (d) and (e) can be selected in a manner known to a person skilled in the art, so that opaque or transparent dental materials are obtained.

The dyes mentioned under (f) are soluble dyes or pigment dyes. If the dental materials according to the invention are used for applications in dental medicine and dental technology, they preferably contain food dyes and/or iron oxide dental dyes as dyes. Dye pastes made of polysiloxane or mineral oil/dye formulations are also suitable for this purpose.

Zeolites, anhydrous aluminium sulfate, molecular sieve, silica gel, and blue gel can be used as moisture binders (g).

As inhibitors (h), the two-component dental materials according to the invention can contain all types of divinyl disiloxanes having the general formula IX
CH₂═C H—SiR₂O—SiR₂—CH═CH₂
where R refers to the same or different, optionally substituted hydrocarbon radicals, such as alkyl, alkenyl and alkinyl groups.

Particularly preferably, divinyl tetraalkyl disiloxanes and/or divinyl tetramethyl disiloxane are used as inhibitors. Alternatively or in addition to these compounds, cyclic siloxanes that contain vinyl groups, for example tetravinyl tetramethyl cyclotetrasiloxane, or organic hydroxyl compounds that contain end-position double or triple bonds, e.g. ethinyl cyclohexanol, can be used as inhibitors.

The solid or liquid QM resins that contain vinyl groups, Si—H and/or Si—OR (where R═H or alkyl) groups, named under (i), are characterized in that they contain tetrafunctional SiO_{4/2} as the Q unit, and monofunctional R₃SiO_{1/2} as the M component, wherein R can be equal to vinyl, methyl, ethyl, phenyl. Furthermore, trifunctional RSiO_{3/2} can also be present as T units, and bifunctional R₂SiO_{2/2} can be present as D units, where R has the same meaning as indicated above. These QM resins can be present dissolved in organopolysiloxanes having two or more vinyl groups in the molecule, and a viscosity from 21 to 350,000 mPa.s. The vinyl group content of the stated QM resins preferably lies in the range of 0.1 to 8 mmol/g. The SiH content preferably lies between 0 and 15 mmol/g, particularly preferably between 0 and 10 mmol/g and more preferably between 0 and 1 mmol/g. The Si—OR content preferably lies between 0 and 0.5 mmol/g. The low SiOH content of the QM resins is preferred because this low SiOH prevents gasification by means of hydrogen development. The proportion of volatile components of the QM resins should also be as low as possible, so that the dimensional stability is not impaired.

The compounds (j) that might be used are preferably composed of organopolysiloxanes having two or more vinyl groups in the molecule and a viscosity of 21 to 350,000 mPa.s, as well as the reinforcing fillers named under (d). Particularly preferably, compounds that are hydrophobized in situ, using modification aids, e.g. hexamethyl disilazane, are used.

The components (k) that might be used as a surfactant, emulsifier and/or stabilizer, can be anionic surfactants, particularly alkyl sulfates, alkyl benzol sulfonates and phosphates, cationic surfactants, particularly tetraalkyl ammonium halogenides, non-ionic surfactants, particularly alkyl and alkyl phenyl polyalkyl alkylene oxides, fatty acid alkoxylates, fatty alcohol alkyloxylates and their alkyl ethers and alkyl esters, fatty acid alkylol amide, saccharose fatty acid esters, trialkylamine oxides, silicone surfactants, or fluorine surfactants, as well as amphoteric surfactants, particularly sulfated or oxyethylated condensation products of alkylene phenols and formaldehyde, ethylene oxide/propylene oxide block polymerizates and/or modified polysiloxanes. Furthermore, the surfactants can also contain functional groups such as —OH—, —CH═CH₂, —OCO—(CH₃)C═CH₂, and SiH. Beyond this list, of course, all other compounds known to a person skilled in the art for this purpose can be used, even if these compounds are not preferred.

As radio-opaque substances (1), the two-component dental materials according to the invention can have, for example, types of glass that contain barium, strontium, lanthane, or zinc; barium sulfate; zirconium dioxide; lanthane oxide; or ceramic filler compositions that contain oxides of lanthane, hafnium, or rare earth metals. Furthermore, complex heavy metal fluorides having the general formula M^{II}M^{IV}F₆ or YF₃ can be used for this purpose, wherein M^{II} is particularly a calcium, strontium, or barium ion, and M^{IV} is particularly a titanium, zirconium, or hafnium ion. Furthermore, atoms or atom groups bound to the silicone polymer, which possess radio-opaque properties, for example iodine bound to silicon, can be used as radio-opaque substances.

The H₂ absorbers/adsorbers named under (m) are preferably microfine palladium or platinum or their alloys, which might be contained in alumosilicates. Furthermore, substances that eliminate or reduce H₂ development can also be used, such as, for example, 3-methyl-1-butin-3-ol and CH₃Si [O—C (CH₃)₂—C≡CH]₃.

The total content of additives and processing aids (f) to (h) and (k) to (n) preferably lies between 0 and 10, particularly preferably between 0 and 7, and very particularly preferably between 0 and 5 wt.-%.

Preferably, the dental material according to the invention, i.e. the mixture of the two components of the addition-crosslinked two-component system contains the substances listed below, in the amount ranges indicated, in each instance, in wt.-%, with reference to the total dental material:
(a₁) 1 to 90 wt.-%, particularly preferably 5 to 75 wt.-%, and very particularly preferably 10 to 60 wt.-% of at least one compound (a) having at least one rigid and/or voluminous group as well as at least two vinyl-functional functional silyl groups in the molecule, and an Si vinyl content of 0.5 to 10 mmol/g, preferably an Si vinyl content of 1 to 10 mmol/g, and particularly preferably an Si vinyl content of 2 to 10 mmol/g,
(a₂) 0 to 40 wt.-%, particularly preferably 0 to 30 wt.-%, and very particularly preferably 0 to 20 wt.-% of at least one organopolysiloxane having at least two vinyl groups in the molecule, and an Si vinyl content of 0.5 to 10 mmol/g, preferably an Si vinyl content of 1 to 10 mmol/g, and particularly preferably an Si vinyl content of 2 to 10 mmol/g, as well as a viscosity of 21 to 350,000 mPa.s,
(b₁) 0 to 90 wt.-%, particularly preferably 5 to 75 wt.-%, and very particularly preferably 10 to 60 wt.-% of an organohydrogen silicon compound having at least one rigid and/or voluminous group as well as at least two silyl groups having at least two, preferably at least three SiH groups, and an SiH content of 0.1 to 15 mmol/g, preferably 4 to 15 mmol/g, and particularly preferably 7 to 15 mmol/g,
(b₂) 0 to 50 wt.-%, particularly preferably 0 to 40 wt.-%, and very particularly preferably 0 to 30 wt.-% of an organohydrogen polysiloxane having at least two, preferably at least three SiH groups, and an SiH content of 0.1 to 15 mmol/g, preferably 4 to 15 mmol/g, particularly preferably 7 to 15 mmol/g, and very particularly preferably 7 to 13 mmol/g, as well as a viscosity between 5 and 2,000 mPa.s, whereby the sum of the weight percents of the compounds (b₁) and (b₂) is at least 1 wt.-%,
(c) 0.00001 to 0.2 wt.-%, preferably 0.0005 to 0.1 wt.-% of at least one catalyst for accelerating the hydrosylilation reaction, with reference to pure metal,
(d) 0 to 50 wt.-%, particularly preferably 0.1 to 40 wt.-%, and very particularly preferably 0.5 to 35 wt.-% of reinforcing fillers having a BET surface of at least 50 m²/g,
(e) 0 to 90 wt.-%, particularly preferably 0 to 80 wt.-%, and very particularly preferably 0 to 75 wt.-% of non-reinforcing fillers having a BET surface of less than 50 m²/g and an average grain size of at least 0.1 μm,
(f) 0 to 5 wt.-%, preferably 0 to 2 wt.-% of at least one dye,
(g) 0 to 30 wt.-%, preferably 0 to 5 wt.-% of moisture binder(s),
(h) 0 to 1 wt.-%, preferably 0 to 0.6 wt.-%, and particularly preferably 0 to 0.1 wt.-% inhibitors,
(i) 0 to 40 wt.-%, particularly preferably 0 to 30 wt.-%, and very particularly preferably 0 to 20 wt.-% of at least one QM resin that contains vinyl groups and/or SiH and/or SIOR (where R═H or alkyl), having an SiH content of 0 to 15 mmol/g, particularly preferably between 0 and 10 mmol/g and more preferably between 0 to 1 mmol/g, and/or an SiOR content of 0 to 0.5 mmol/g,
(j) 0 to 80 wt.-%, preferably 0 to 50 wt.-% of compounds of organopolysiloxanes that contain vinyl groups, and reinforcing fillers,
(k) 0 to 10 wt.-%, preferably 0 to 5 wt.% of surfactants, emulsifiers and/or stabilizers,
(l) 0 to 90 wt.-%, preferably 0 to 80 wt.-% of radio-opaque substances,
(m) 0 to 20 wt.-%, preferably 0 to 10 wt.-%, of H₂ absorbers/adsorbers or substances that reduce or eliminate H₂ development, as well as
(n) 0 to 20 wt.-%, preferably 0 to 15 wt.-%, of other aids and additives.

The two-component dental materials according to the invention, in the fully vulcanized state, have a high Shore D final hardness and/or great flexural strength and/or a high modulus of elasticity in the bending test. At the same time these materials have a short setting time. These compositions are therefore suitable for all uses in dental medicine and dental technology in which a high final hardness and a low elasticity of the material are required. In particular, the materials according to the invention are suitable for bite registration, fixation, taking tooth impressions, for positioning, as cement, as temporary crowns, as bridge material, as temporary as well as permanent filling material, for keying, for restoration, for transfer of brackets in gnathic orthopedics, and for replacement of fillings for grinding in the occlusion on the metal. In combination with dental glass (average grain diameter from 10 to 0.1 μm) and nano-fillers (average grain diameter less than 100 nm), excellent mechanical properties and optical properties, with regard to translucence, can be achieved, such as those required, for example, for the production of temporary crowns and bridges, and for filling material.

Another object of the present invention is crosslinking dental materials obtained by way of hydrosilylation, containing
(a) one or more compounds having vinyl groups in the molecule,
(b) at least one organohydrogen silicon compound, and
(c) at least one catalyst,
   wherein the at least one compound (a) and/or the at least one compound (b) comprises,
as the first structural unit, at least one voluminous and/or rigid group, as well as
as the second structural unit, at least two alkenyl-functional or at least two, preferably at least three hydrogen-functional silyl units having the general formula I 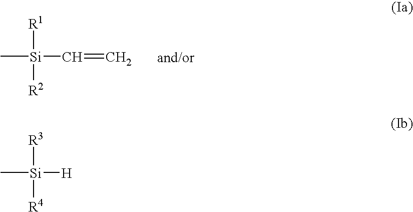
   where R¹, R², independent of one another, are selected from the group consisting of alkyl groups, alkenyl groups, aryl groups, aralkyl groups, alkylaryl groups, halogenated alkyl groups, halogenated aryl groups, halogenated aralkyl groups, halogenated alkylaryl groups, cyanoalkyl groups, siloxy groups, cycloalkyl groups, and cycloalkenyl groups, wherein alkyl groups are preferred and methyl groups are particularly preferred, and
R³, R⁴, independent of one another, are H or R¹, wherein alkyl groups are preferred and methyl groups are particularly preferred,
wherein the first and second structural unit are bound
(i) directly,
(ii) by way of an oxygen atom,
(iii) by way of a spacer group, or
(iv) by way of a spacer group according to (iii), which is bound to the first structural unit by way of an oxygen atom,
and the voluminous and/or rigid group E and/or the second structural unit is/are a linear or cyclic monosiloxane, oligosiloxane, or polysiloxane unit.

Surprisingly, these compositions also cure to form masses having excellent mechanical properties, particularly outstanding strength and modulus of elasticity. With regard to the particular embodiments and other optional components (d) to (n), the same applies as for the compounds (a) and (b) that do not contain any cyclic oligosiloxane group. Examples of particularly preferred compounds having cyclic oligosiloxane groups are: 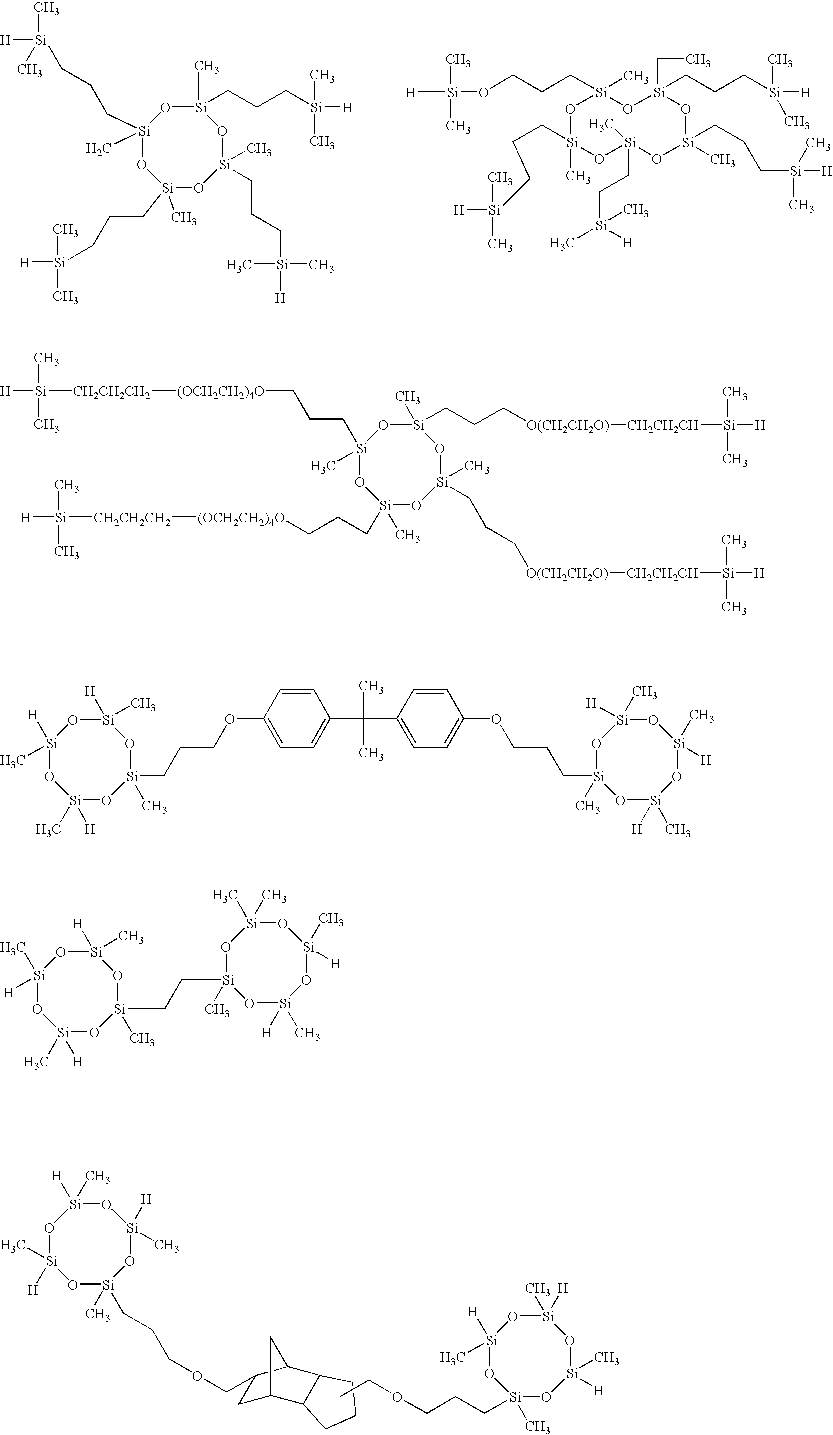 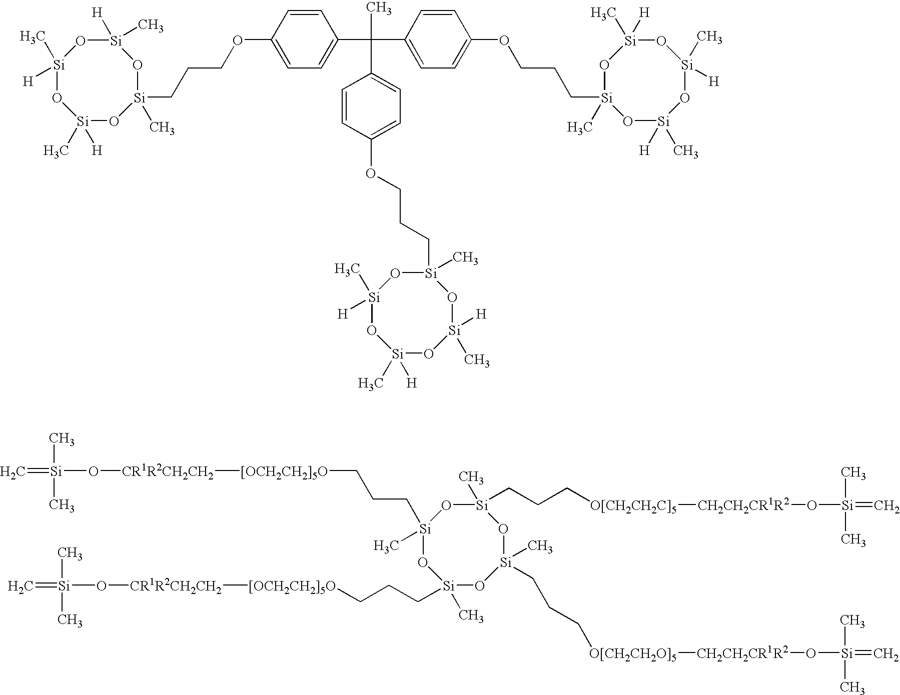
where s=0 to 10 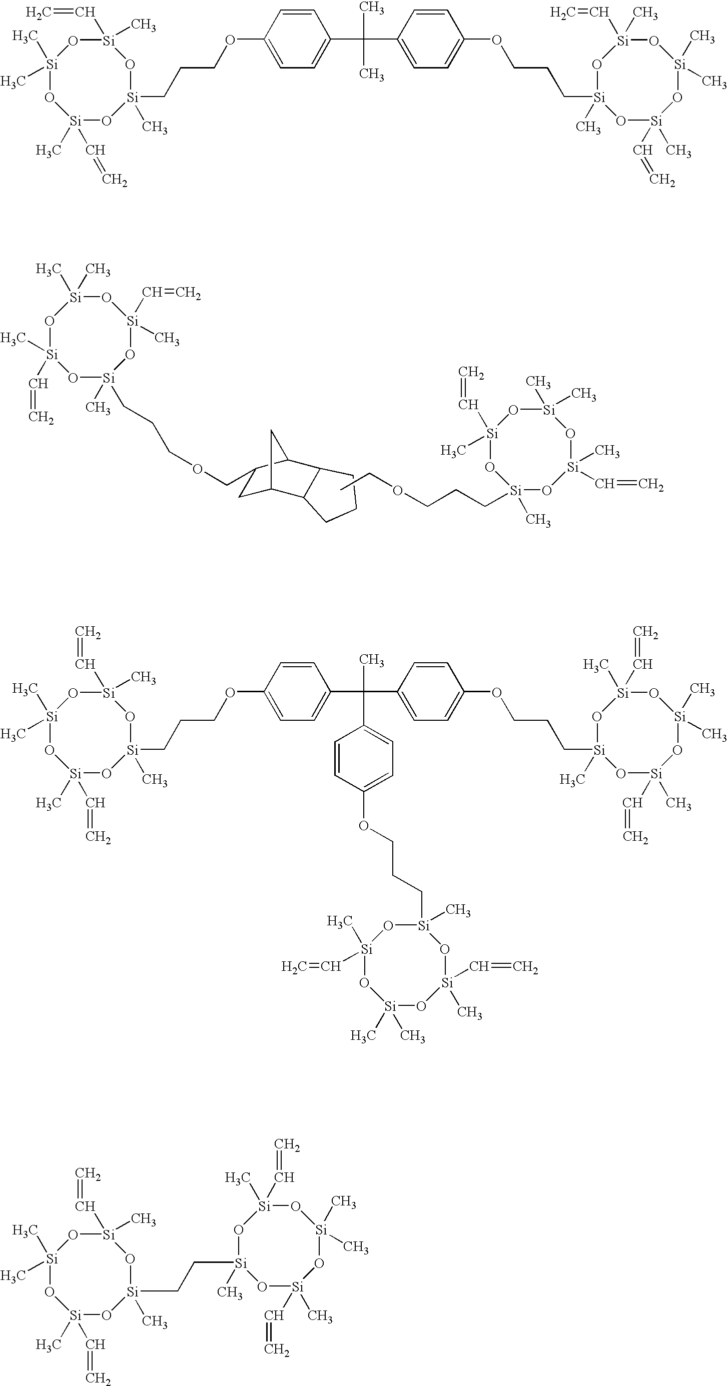 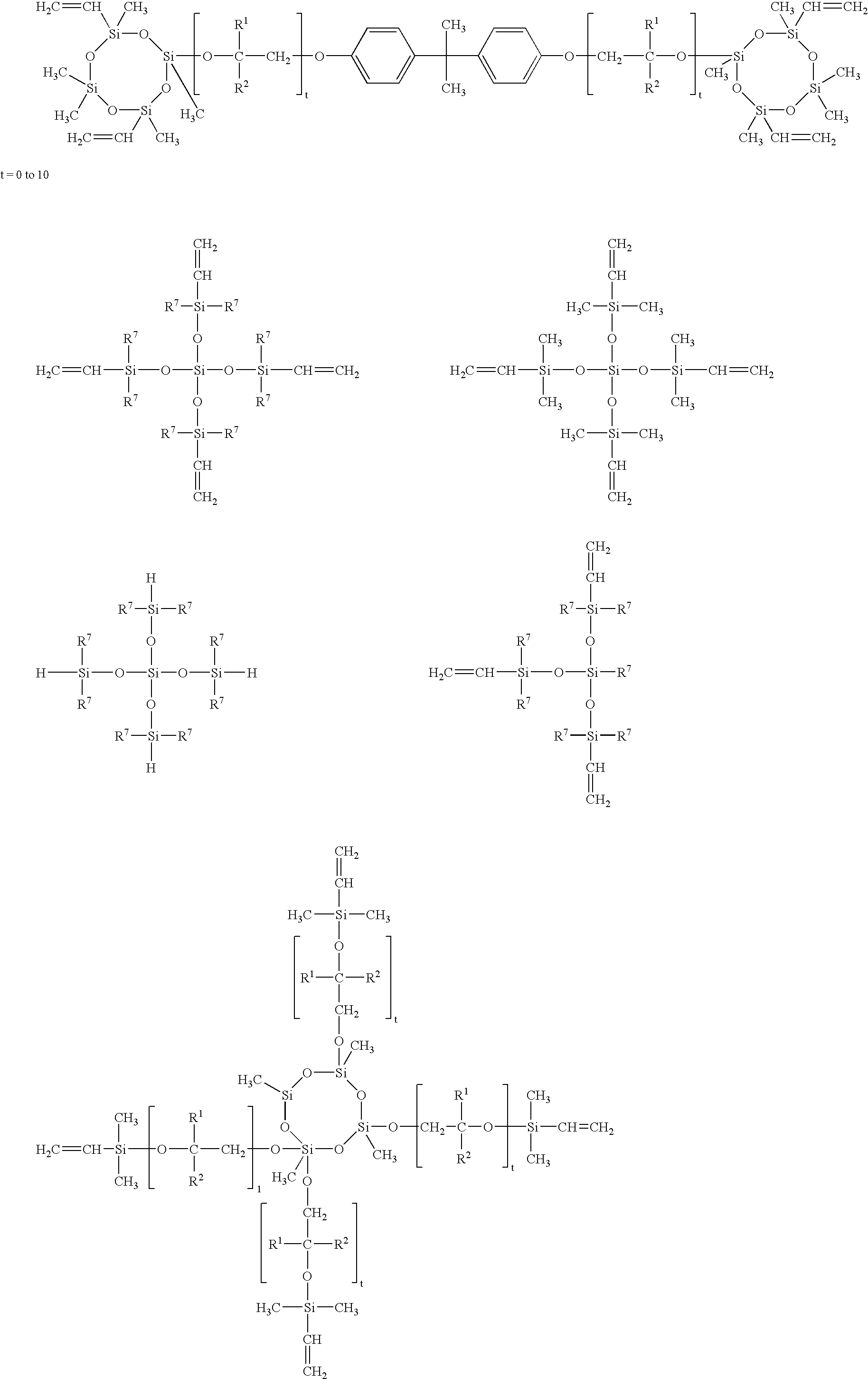 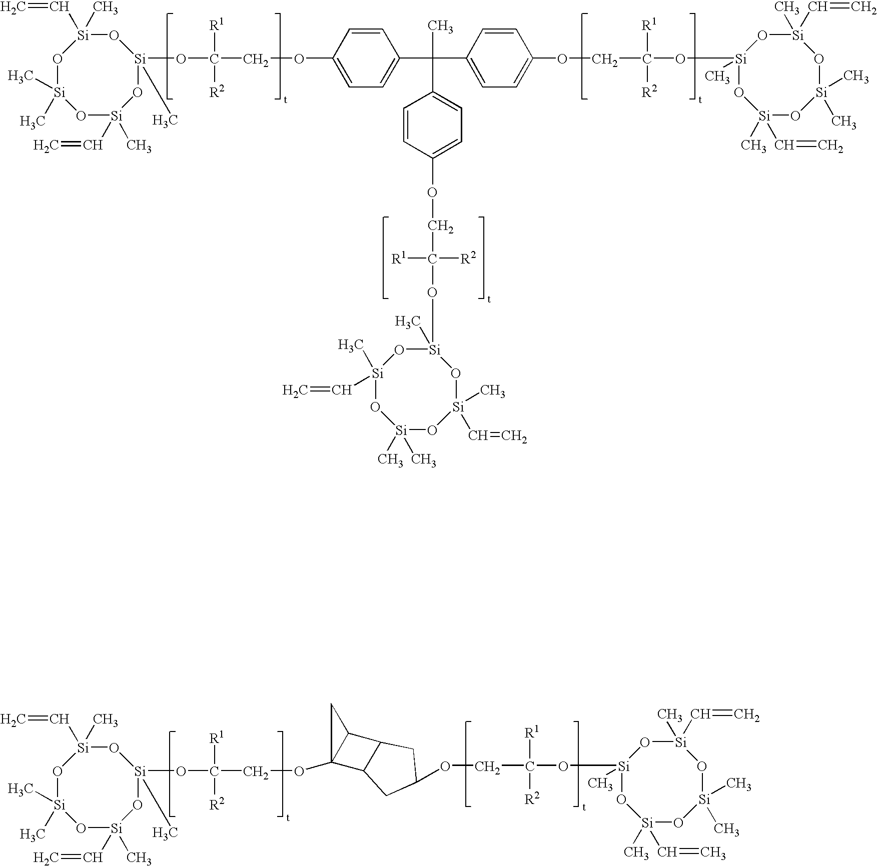
where R¹ and R² are as defined in formula I, and where R⁷ is as defined in formula VII, in each instance.

In the following, the invention will be explained using examples that demonstrate the idea of the invention, but do not limit it.

### SYNTHESIS EXAMPLE 1

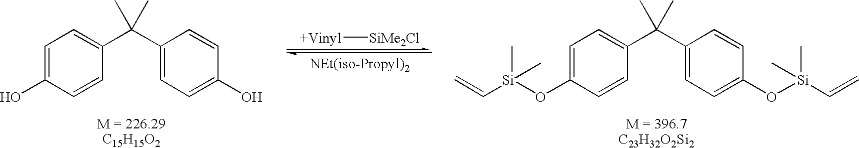

25 g (110 mmol) bisphenol A were dissolved in 250 ml triethylene glycol dimethyl ether/tert.-butyl toluene (1:1) and subsequently, 75 ml NEt(iso-propyl)₂ were added to this solution. When 35.8 ml (262 mmol) vinyl-SiMe₂Cl were dripped in, a white precipitate formed. After 24 hours at 90° C oil bath temperature, the precipitate was filtered off and the solvent was drawn off by way of a U tube, under vacuum, at 120° C.

42 g of product, corresponding to a yield of 96%, were obtained.

### SYNTHESIS EXAMPLE 2

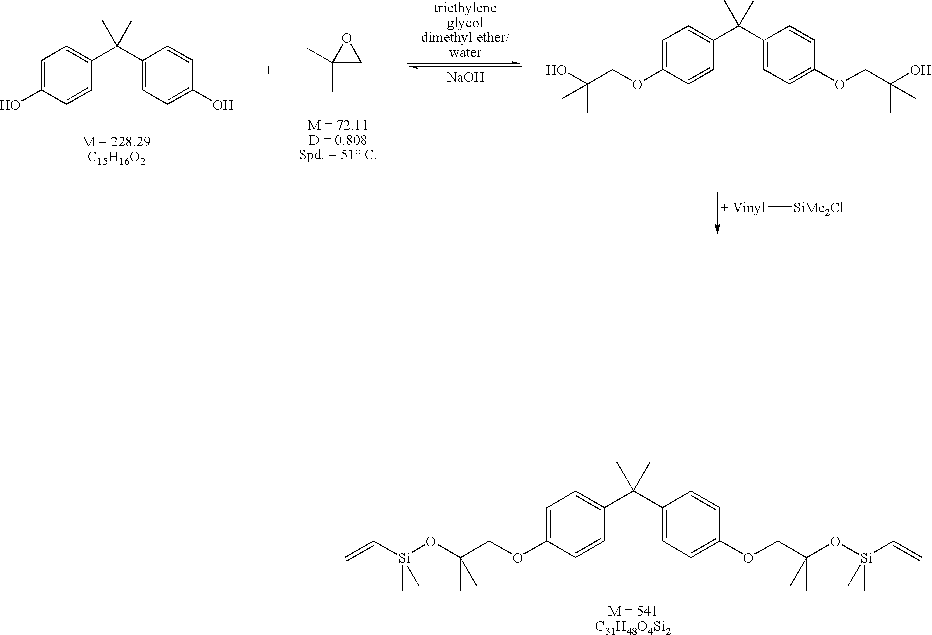

25 g (110 mmol) bisphenol A were dissolved in 250 ml triethylene glycol dimethyl ether, and a concentrated NaOH solution (11 mmol) was added. After adding 100 ml water and 39 ml 2,2-dimethyloxiran, the mixture was stirred for 2 days at 95° C. The solvents were drawn off by way of a U tube, in a vacuum, at 120° C. Subsequently, the residue was dissolved in triethylene glycol dimethyl ether/tert.-butyl toluene and 75 ml NEt(iso-propyl)₂ were added. When 35.8 ml (262 mmol) vinyl-SiMe₂Cl were dripped in, a white precipitate formed. After 24 hours at 90° C. oil bath temperature, the precipitate was filtered off and the solvent was drawn off by way of a U tube, under vacuum, at 120° C.

56 g of a brown, viscous fluid were obtained as the product, corresponding to a yield of 95%.

### SYNTHESIS EXAMPLE 3

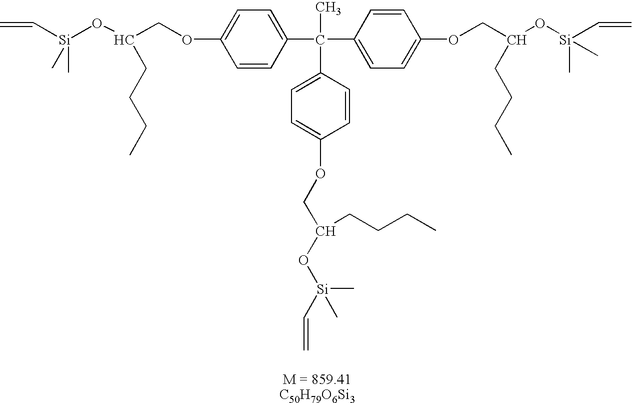

50 g (163 mmol) 1,1,1-tris-(4-hydroxyphenyl)ethane were dissolved in 300 ml tetraethylene glycol dimethyl ether, and a concentrated NaOH solution (25 mmol) was added to this solution. After adding 100 ml water and 88 ml (734 mmol) butyloxiran, the mixture was stirred for 2 days at 100° C. The solvents were drawn off by way of a U tube, in a vacuum, at 140° C. 79 g (80%) of a viscous fluid were obtained, which were subsequently dissolved in 600 ml tert.-butyl toluene and 168 ml NEt(iso-propyl)₂.

80 ml (587 mmol) vinyl dimethyl chlorosilane were added to this solution, and it was stirred for 24 hours at 90° C. Afterwards, the mixture was cooled to room temperature, the precipitate was filtered off, and the solvent was drawn off in a vacuum, at 120° C. 133 g of a brown, viscous compound, still capable of flowing, were obtained as the product, corresponding to a yield of 95%.

### SYNTHESIS EXAMPLE 4

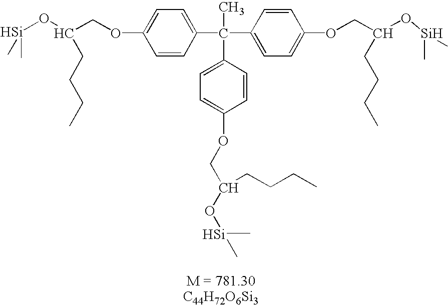

50 g (163 mmol) 1,1,1-tris-(4-hydroxyphenyl)ethane were dissolved in 300 ml tetraethylene glycol dimethyl ether, and a concentrated NaOH solution (25 mmol) was added to this solution. After adding 100 ml water and 88 ml (734 mmol) butyloxiran, the mixture was stirred for 2 days at 100° C., under reflux. The solvents were drawn off by way of a U tube, in a vacuum, at 140° C. 79 g (80%) of a viscous fluid were obtained, which were subsequently dissolved in 600 ml tert.-butyl toluene and 168 ml NEt(iso-propyl)₂.

64 ml (587 mmol) dimethyl chlorosilane were added to this solution, and it was stirred for 24 hours at 80° C. Afterwards, the mixture was cooled to room temperature, the precipitate was filtered off, and the solvent was drawn off in a vacuum, at 120° C.

114 g of a yellowish, viscous compound were obtained as the product, corresponding to a yield of 90%.

### SYNTHESIS EXAMPLE 5

### Production of 4,4′-bis(vinyl dimethyl silyl)biphenyl

### 1^{st} Step: Synthesis of 4,4′-bis(ethoxy dimethyl silyl)biphenyl

4.81 g (0.198 mol) dried magnesium and 26.81 g (0.181 mol) Me₂Si(OEt)₂ were presented in a glass flask, and 10 ml tetrahydrofurane (THF) were added to this mixture. Subsequently, approximately 1 ml of a solution of 4,4′-dibromobiphenyl A (26.86 g, 0.0861 mol), dissolved in THF (25 ml), were added under a nitrogen atmosphere. The mixture was heated in order to start the reaction. After 20 minutes, another milliliter of the solution was added to the mixture. During the constant provision of heat, the remaining solution of 4,4′-dibromobiphenyl A in THF was added, drop by drop, over a time period of 45 minutes. After the solution had been added, the mixture was heated, with simultaneous reflux of THF.

The consumption of the starting material was checked by means of gas chromatography (GC). THF and salt that formed were removed. By means of distillation under reduced pressure, 16.78 g (0.049 mol) 4,4′-bis(ethoxy dimethyl silyl)-biphenyl A, corresponding to a yield of 57%, were obtained as a colorless, transparent fluid.

### 2^{nd} Step: Synthesis of 4,4′-bis(vinyl dimethyl silyl) biphenyl

In a glass flask, 10 ml THF were added to 17.54 g (51 mmol) 4,4′-bis(ethoxy dimethyl silyl)-biphenyl. Subsequently, 108 ml (108 mmol) of a 1.0 M solution of CH₂═CHMgBr in THF was added to the mixture, drop by drop, at room temperature, over a time period of 30 minutes under a nitrogen atmosphere. After addition of the Grignard compound in THF, heating with reflux took place over a time period of 5 hours. After the mixture had been incubated overnight at room temperature, the consumption of the starting material and the formation of the desired product were checked by means of gas chromatography (GC).

10 ml methanol were added to the mixture obtained, in order to decompose excess CH₂═CHMgBr, and THF and salt that formed were removed. By means of distillation under reduced pressure, the compound was isolated as a colorless, transparent fluid. The yield corresponded to approximately 30-50%.

### EXAMPLE 1

### According to the Invention

In a closed kneader, 36 parts of the compound from Synthesis Example 1 were homogenized with 61 parts quartz powder having an average grain size of 10 μm, two parts of a highly disperse, hydrophobized silicic acid produced pyrogenically, having a BET surface of 170 m²/g, and one part of a platinum catalyst having a content of pure platinum of 1%, for 1.5 hours, and afterwards de-gassed in a vacuum for 15 minutes.

A paste demonstrating medium flow (ISO 4823) was obtained. The paste represents Component A of the two-component silicone material according to the invention, according to a first exemplary embodiment. After storage at 23° C. for one month, the viscosity and the reactivity were in the required range.

### EXAMPLE 2

### According to the Invention

In a closed kneader, 6 parts of the compound from Synthesis Example 1 were homogenized with 30 parts of a polymethyl hydrogen siloxane having a viscosity of 30 mPa.s (measured at 20° C.) as well as an SiH content of 9.3 mmol/g (Silopren® U930), 63 parts quartz powder having an average grain size of 10 μm, and one part of a highly disperse, hydrophobized silicic acid produced pyrogenically, having a BET surface of 170 m²/g, for 1.5 hours, and afterwards de-gassed in a vacuum for 15 minutes.

A paste demonstrating medium flow (ISO 4823) was obtained. The paste represents Component B of the two-component silicone material according to the invention, according to a first exemplary embodiment. After storage at 23° C. for one month, the viscosity and the reactivity were in the required range.

### EXAMPLE 3

### According to the Invention

50 parts of Component A described in Example 1, and 50 parts of Component B described in Example 2 were pressed out of a cartridge (Mixpac) and homogeneously mixed using a static mixer (Mixpac).

The product remained workable for approximately 60 seconds at room temperature, and cured completely within about three minutes after the start of mixing, at a temperature of 35° C. Hard, difficult to compress shaped bodies were obtained as the vulcanizate, and these bodies could be milled.

After 24 hours of storage of the sample bodies in water, at 37° C., the following mechanical properties were determined:

| | |
|---|---|
| Shore D hardness (DIN 53505): | 70 |
| Modulus of elasticity in the three-point | 902 MPa |
| bending test (ISO 10477): | |
| Flexural strength (ISO 10477): | 20.7 MPa |

The aforementioned measurement values are average values, in each instance, from the testing of five sample bodies.

The modulus of elasticity in the three-point bending test was calculated as a secant modulus between 0.05% and 0.25% bending, according to the following formula:
modulus of elasticity=[Lᵥ^{3*}(X_{H}−X_{L})]/(4*delta−L*b₀*a₀³
The flexural strength was calculated according to the following formula:
flexural strength=[(1.5*Lᵥ)/(b₀*a₀²)]*F
where:
a₀: sample thickness
b₀: sample width
L_{V}: support width
X_{H}: force at the end of determining the modulus of elasticity
X_{L}: force at the start of determining the modulus of elasticity
delta L: bending between X_{H} and X_{L} and
F: force at fracture of the sample
   A flat/strip sample having the following dimensions was used as the sample body:
sample thickness a₀=2 mm, sample width b₀=2 mm, sample length=25 mm.
   The support width was Lv=20 mm.

### EXAMPLE 4

### According to the Invention

In a closed kneader, 36 parts of the compound from Synthesis Example 2 were homogenized with 61 parts quartz powder having an average grain size of 0.5 μm, two parts of a highly disperse, hydrophobized silicic acid produced pyrogenically, having a BET surface of 170 m²/g, and one part of a platinum catalyst having a content of pure platinum of 1%, for 1.5 hours, and afterwards de-gassed in a vacuum for 15 minutes.

A paste demonstrating medium flow (ISO 4823) was obtained. The paste represents Component A of the two-component silicone material according to the invention, according to a second exemplary embodiment. After storage at 23° C. for one month, the viscosity and the reactivity were in the required range.

### EXAMPLE 5

### According to the Invention

In a closed kneader, 33 parts of a polymethyl hydrogen siloxane having a viscosity of 30 mPa.s (measured at 20° C.) as well as an SiH content of 9.3 mmol/g were homogenized with 65 parts quartz powder having an average grain size of 0.5 μm, and two parts of a highly disperse, hydrophobized silicic acid produced pyrogenically, having a BET surface of 170 m²/g, for 1.5 hours, and afterwards de-gassed in a vacuum for 15 minutes.

A paste demonstrating medium flow (ISO 4823) was obtained. The paste represents Component B of the two-component silicone material according to the invention, according to a first exemplary embodiment. After storage at 23° C. for one month, the viscosity and the reactivity were in the required range.

### EXAMPLE 6

### According to the Invention

50 parts of Component A described in Example 4, and 50 parts of Component B described in Example 5 were pressed out of a cartridge (Mixpac) and homogeneously mixed using a static mixer (Mixpac).

The product remained workable for approximately 60 seconds at room temperature, and cured completely within about three minutes after the start of mixing, at a temperature of 35° C. Hard, difficult to compress shaped bodies were obtained as the vulcanizate, and these could be milled.

After 24 hours of storage of the sample bodies in water, at 37° C., the following mechanical properties were determined:

| | |
|---|---|
| Shore D hardness (DIN 53505): | 75 |
| Modulus of elasticity in the three-point | 1,074 MPa |
| bending test (ISO 10477): | |
| Flexural strength (ISO 10477): | 19.4 MPa |

### EXAMPLE 7

### According to the Invention

In a closed kneader, 35 parts of the compound from Synthesis Example 3 were homogenized with 64 parts quartz powder having an average grain size of 3 μm, two parts of a highly disperse, hydrophobized silicic acid produced pyrogenically, having a BET surface of 170 m²/g, and one part of a platinum catalyst having a content of pure platinum of 1%, for 1.5 hours, and afterwards de-gassed in a vacuum for 15 minutes.

A paste demonstrating medium flow (ISO 4823) was obtained. The paste represents Component A of the two-component silicone material according to the invention. After storage at 23° C. for one month, the viscosity and the reactivity were in the required range.

### EXAMPLE 8

### According to the Invention

In a closed kneader, 10 parts of the compound from Synthesis Example 3 were homogenized with 67 parts quartz powder having an average grain size of 3 μm, and two parts of a highly disperse, hydrophobized silicic acid produced pyrogenically, having a BET surface of 170 m²/g, and 21 parts of a polymethyl hydrogen siloxane having a viscosity of 30 mPa.s (measured at 20° C.) and an SiH content of 9.3 mmol/g, for 1.5 hours, and afterwards de-gassed in a vacuum for 15 minutes.

A paste demonstrating medium flow (ISO 4823) was obtained. The paste represents Component B of the two-component silicone material according to the invention. After storage at 23° C. for one month, the viscosity and the reactivity were in the required range.

### EXAMPLE 9

### According to the Invention

50 parts of Component A described in Example 7, and 50 parts of Component B described in Example 8 were pressed out of a cartridge (Mixpac) and homogeneously mixed using a static mixer (Mixpac).

The product remained workable for approximately 60 seconds at room temperature, and cured completely within about two minutes after the start of mixing, at a temperature of 35° C. Hard, difficult to compress shaped bodies were obtained as the vulcanizate, and these bodies could be milled.

After 24 hours of storage of the sample bodies in water, at 37° C., the following mechanical properties were determined:

| | |
|---|---|
| Shore D hardness (DIN 53505): | 83 |
| Modulus of elasticity in the three-point | 2,226 MPa |
| bending test (ISO 10477): | |
| Flexural strength (ISO 10477): | 31.7 MPa |

### COMPARISON EXAMPLE 1

### Not According to the Invention

A commercially available bite registration material on the basis of addition-crosslinking vinyl polysiloxanes was mixed in accordance with the manufacturer's instructions and brought to binding.

The product remained workable at room temperature for approximately 30 seconds and cured completely within about two minutes after the start of mixing, at a temperature of 35° C.

Shaped bodies were obtained as the vulcanizate, and these bodies could be milled well.

After 24 hours of storage of the sample bodies in water, at 37° C., the following mechanical properties were determined:

| | |
|---|---|
| Shore D hardness (DIN 53505): | 43 |
| Modulus of elasticity in the three-point | 38 MPa |
| bending test (ISO 10477): | |
| Flexural strength (ISO 10477): | 8.7 MPa |

The determination and calculation of the modulus of elasticity in the three-point bending test and of the flexural strength took place as described in Example 3.

This example illustrates that bite registration materials on the basis of addition-crosslinking vinyl polysiloxanes according to the state of the art have a noticeably lower hardness, i.e. a significantly lower modulus of elasticity.

The results of the individual examples and comparison examples are summarized in Table 1.

TABLE 1

| | Modulus of | | | | |
|---|---|---|---|---|---|
| | elasticity⁽¹⁾ | | | | |
| | in the three- | | | | |
| | point bending | Flexural | Shore D | Shore A | |
| Example | test | strength⁽¹⁾ | hardness⁽¹⁾ | hardness⁽¹⁾ | Setting time |
| Example 1 | 902 MPa | 20.7 MPa | 70 | >95 | 3.0 min. |
| Example 6 | 1,074 MPa | 19.4 MPa | 75 | >95 | 3.0 min. |
| Example 9 | 2,226 MPa | 31.7 MPa | 83 | >95 | 2.0 min. |
| Comparison example 1 | 38 MPa | 8.7 MPa | 43 | >95 | 2.0 min. |

⁽¹⁾after 24 hours at 37° C.

Although several embodiments of the present invention have been shown and described, it is to be understood that many changes and modifications may be made thereunto without departing from the spirit and scope of the invention as defined in the appended claims.

1. A two-component, dental material addition-crosslinking by way of hydrosilylation, comprising:
(a) at least one compound having at least two vinyl groups in the molecule;
(b) at least one organohydrogen silicon compound; and
(c) at least one catalyst;
wherein the at least one compound (a) or the at least one compound (b) comprises a first structural unit and a second structural unit;
said first structural unit comprising, at least one voluminous or rigid group; and
said second structural unit comprising at least two alkenyl-functional or at least two hydrogen-functional silyl units having the general formula I for 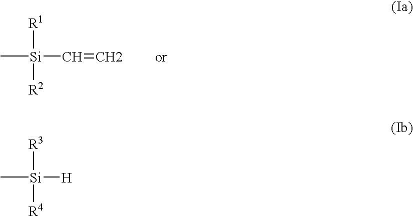
where R¹, R², independent of one another, are selected from the group that consists of alkyl groups, alkenyl groups, aryl groups, aralkyl groups, alkylaryl groups, halogenated alkyl groups, halogenated aryl groups, halogenated aralkyl groups, halogenated alkylaryl groups, cyanoalkyl groups, siloxy groups, cycloalkyl groups, and cycloalkenyl groups, and
R³, R⁴, independent of one another, are H or R¹;
wherein the second structural unit is bound to the first structural unit
(i) directly;
(ii) by way of an oxygen atom;
(iii) by way of a spacer group; or
(iv) by way of a spacer group bound to the first structural unit by way of an oxygen atom.

2. The dental material according to claim 1 wherein said first structural unit is selected from the group that consists of tertiary alkyl, quaternary alkyl, cycloalkyl, cycloalkenyl, aryl, aralkyl, alkylaryl, halogen-substituted tertiary alkyl, halogen-substituted quaternary alkyl, halogenated aryl, halogenated aralkyl, and halogenated alkylaryl; and said second structural unit comprises at least three hydrogen-functional silyl units having the general formula I wherein R¹, R², R³ and R⁴ are alkyl groups.

3. The dental material according to claim 1 wherein said first structural unit is selected from the group that consists of aromatic and non-aromatic mono, bis, oligo, polycyclic groups, bisphenol A, bisphenol B, bisphenol F groups, 1,1,1-tris(4-hydroxyphenyl)alkane groups, norbornane groups, adamantane groups, and pentaerythrite groups.

4. The dental material according to claim 1 wherein R¹, R², R³ and R⁴ are methyl groups.

5. The dental material according to claim 1 wherein each of said at least one compound (a) and said at least one compound (b) comprises said first and second structural units, said at least one compound (a) comprising at least two vinyl groups at a first distance of at least 0.3 nm, and said at least one compound (b) comprising at least two SiH groups at a second distance of at least 0.3 nm.

6. The dental material according to claim 5 wherein each of the first and second distances is at least 0.5 nm.

7. The dental material according to claim 5 wherein each of the first and second distances is at least 0.7 nm.

8. The dental material according to claim 1 wherein the first structural unit is an aromatic mono, bis, oligo or polycyclus or a non-aromatic mono, bis, oligo or polycyclus.

9. The dental material according to claim 8 wherein the first structural unit is a bisphenol A group, a bisphenol B group, a bisphenol F group, a 1,1,1-tris(4-hydroxyphenyl)alkane group, a norbornane group, an adamantane group, or a pentaerythrite group.

10. The dental material according to claim 1, wherein the first structural unit is selected from the group that consists of 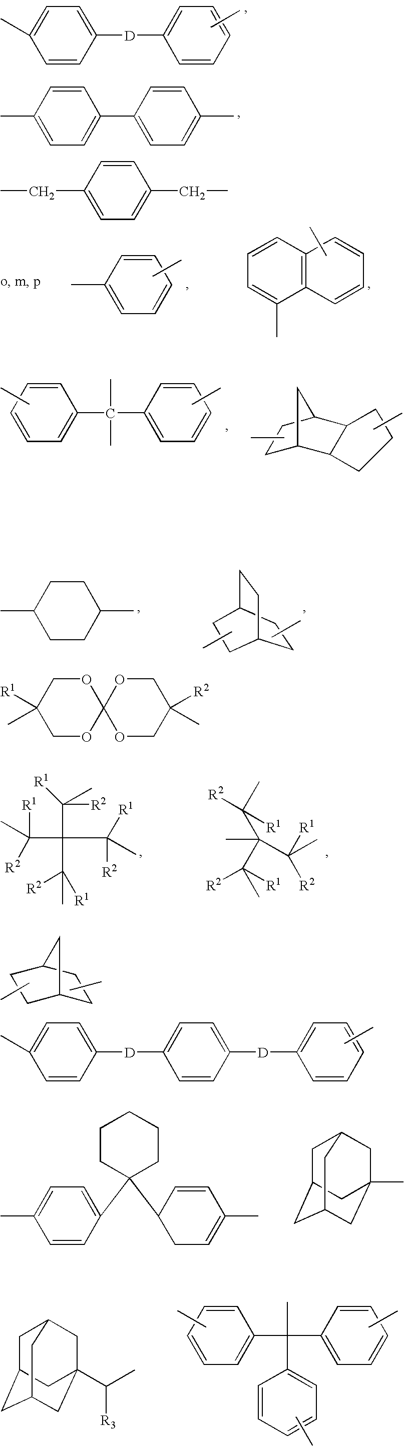
where R¹, R², R³ are as defined in the formula I, and D=CR³R⁴ where R³ and R⁴ are defined in formula Ib, or hydrogen.

11. The dental material as recited in claim 10 wherein D denotes CH₂, C(CH₃)₂, CMePh where Me═CH₃ and Ph═C₆H₅ or CPh₂.

12. The dental material according to claim 1 wherein the first structural unit is bisphenol A, a bisphenol A derivative, or a trisphenol compound according to one of the two general formulas 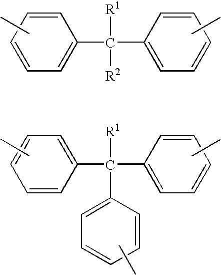
with R¹, R² as defined in the formula I.

13. The dental material according to claim 1 wherein the at least one compound (a) or the at least one compound (b) comprises a substance having the general formula II
H₂C—CH—SiR¹R²-Eₙ₁-SiR¹R²—CH—CH₂ (IIa)
or
H—SiR³R⁴-Eₙ₁-SiR³R⁴—H (IIb),
wherein the radicals R¹, R², R³, R⁴ are as defined in formula I, E denotes the first structural unit, and n₁ is a whole number ≧1.

14. The dental material as recited in claim 13 wherein n₁=1.

15. The dental material according to claim 1 wherein the at least one compound (a) or the at least one compound (b) comprises a substance having the general formula III
H₂C═CH—SiR¹R²—O-Eₙ₁-O—SiR¹R²—CH═CH₂ (IIIa),
or
H—SiR³R⁴—O-Eₙ₁-O—SiR³R⁴—H (IIIb)
wherein the radicals R¹, R², R³, R⁴ are as defined in the formula I, E denotes the first structural unit, and n₁ is a whole number ≧1.

16. The dental material according to claim 1 wherein the at least one compound (a) or the at least one compound (b) comprises a substance having the general formula IV
H₂C═CH—SiR¹R²-A¹ₙ₂-En-A²ₙ₃-SiR¹R²—CH═CH₂ (IVa)
or
H—SiR³R⁴-A¹ₙ₂-Eₙ₁-A²ₙ₃-SiR³R⁴—H (IVb),
wherein the radicals R¹, R², R³, R⁴ are as defined in the formula I, E denotes the first strucutral unit, n₁ is a whole number ≧1, n₂, n₃ are the same or different and are whole numbers ≧1, in each instance, and A¹, A², independent of one another, is a spacer.

17. The dental material according to claim 16 wherein n₁=1.

18. The dental material according to claim 16 wherein the spacers A₁, A², independent of one another, are selected from the group that consists of alkyl, alkoxy, alkenyl, alkylenoxy, cycloalkyl, cycloalkoxy, cycloalkenyl, cycloalkenyloxy, aryl, aralkyl, alkylaryl, aroxy, aralkoxy, alkylaroxy, cyanoalkyl, cyanoalkoxy, halogen-substituted alkyl, halogenated aryl, halogenated aralkyl, halogenated alkylaryl, halogen-substituted alkoxy, halogenated aralkoxy, and halogenated alkylaroxy.

19. The dental material according to claim 16 wherein the spacers A¹, A², independent of one another, are selected from the group that consists of mono, bis, oligo, polyether structural units and polydialkyl siloxane structural units ([—O—SiR¹R²]ₙ, where R¹, R² are as defined in the formula 1, with a repetition unit n of the ether or the siloxane of n=1 to 20.

20. The dental material according to claim 16 wherein the spacers A¹, A², independent of one another, are a substance having the general formula V
—(CR⁵₂—CR⁵₂—O)ₙ₄ (Va)
or
—(CR⁵₂—CR⁵₂—CR⁵₂—CR⁵₂—O)ₙ₄ (Vb),
wherein the radicals R⁵, independent of one another, are H, alkyl, aryl, aralkyl or alkylaryl, and n₄ is a whole number ≧1.

21. The dental material according to claim 20 wherein R⁵ is H or a C₁-C₅ alkyl radical, and n₄ is a whole number between 1 and 20.

22. The dental material according to claim 20 wherein the at least one compound (a) or the at least one compount (b) has a member selected from the group consisting of
OCH₂CH₂—, —O(C H₂)₄—, —O—CR¹R²—CR¹R²—CH₂CH₂CH₂(OCH₂CH₂)₄—, —CH₂—CH₂—CH₂—, —O—C(CH₃)₂—CH₂—, O—CH(CH₃)—CH₂—, —O—CH(C₂H₅)—CH₂—, —O—CH(C₄H₉)—CH₂— O—CH(C₁₀H₂₁)—CH₂—, and
as spacer A.

23. The dental material according to claim 16 wherein the at least one compound (a) or the at least one compound (b) comprises at least one first structual unit and at least one second structural unit, wherein the at least one second structural unit is bound to the at least one first structural unit by way of a spacer bound to the at least one first structural unit by way of an oxygen atom.

24. The dental material according to claim 1 wherein the at least one compound (a) or the at least one compound (b) has one rigid or voluminous group.

25. The dental material according to claim 1 wherein in a fully vulcanized state, the dental material has a Shore D hardness (according to DIN 53505) greater than 35, or a flexural strength of at least 8 MPa, or a modulus of elasticity in a bending test (measured according to ISO 10477) of at least 300 MPa.

26. The dental material according to claim 25 wherein the Shore D hardness is greater than 50, the flexural strength is at least 15 mPa, and the modulus of elasticity is at least 600 MPa.

27. The dental material according to claim 25 wherein the Shore D hardness is greater than 69, the flexural strength is at least 19 MPa, and the modulus of elasticity is at least 900 MPa.

28. The dental material according to claim 1 wherein the at least one compound (a) has an Si vinyl content of 0.5 to 10 mmol/g.

29. The dental material according to claim 28 wherein the at least one compound (a) has an Si vinyl content of 1 to 10 mmol/g.

30. The dental material according to claim 28 wherein the at least one compound (a) has an Si vinyl content of 2 to 10 mmol/g.

31. The dental material according to claim 1 wherein the at least one compound (a) and the at least one compound (b) comprise at least one first structural unit and at least one second structural unit.

32. The dental material according to claim 31, further comprising, in addition to said at least one compound (a) and at least one compound (b), one or more organohydrogen polysiloxanes.

33. The dental material according to claim 32 wherein said one or more organohydrogen polysiloxanes have at least two Si—H groups per molecule and an Si—H content between 0.1 and 15 mmol/g and a viscosity (at 20° C.) of 5 to 2,000 mPa.s.

34. The dental material according to claim 33 wherein said Si—H content is between 4 and 14 mmol/g.

35. The dental material as recited in claim 33 wherein said Si—H content is between 5 and 13 mmol/g.

36. The dental material according to claim 1, wherein the at least one organohydrogen silicon compound (b) is a compound having the general formula VI 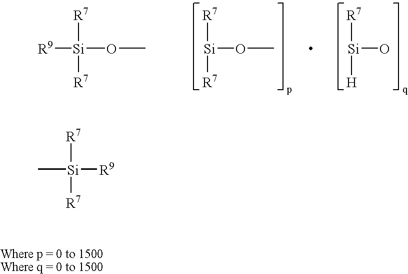
where R⁷=alkyl, aryl, aralkyl, halogen-substituted alkyl, halogen-substituted aryl, cyanoalkyl, cycloalkyl, or cycloalkenyl and
where R⁹═R⁷ or H
with the proviso that at least 2 Si atoms of the formula VI bear an H atom.

37. The dental material as according to claim 36 wherein q=2 to 1500 and R⁷=methyl, ethyl, isopropyl, phenyl, naphthyl, tolyl, xylyl, benzyl, phenylethyl, 3,3,3-trifluoropropyl, chlorophenyl, or difluorophenyl.

38. The dental material according to claim 36 wherein R⁷=methyl.

39. The dental material according to claim 1 wherein the at least one organohydrogen silicon compound (b) is a compound having the general formula VII 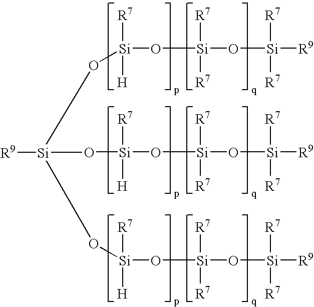
wherein p=0 to 1500, q=0 to 1500, R⁷ alkyl, aryl, aralkyl, halogen-substituted alkyl, halogen-substituted aryl, cyanoalkyl, cycloalkyl, or cycloalkenyl, R⁹═R⁷ or H with the provision that at least 2 Si atoms of the formula VII bear an H atom.

40. The dental material as according to claim 39 wherein q=2 to 1500 and R⁷=methyl, ethyl, isopropyl, phenyl, naphthyl, tolyl, xylyl, benzyl, phenylethyl, 3,3,3-trifluoropropyl, chlorophenyl, or difluorophenyl.

41. The dental material according to claim 39 wherein R⁷=methyl.

42. The dental material according to claim 1 wherein the at least one organohydrogen silicon compound (b) is a compound having the general formula VIII:
[SiO_{4/2}][SiO_{1/2}R¹R²H]ₘ
where m=0.9 to 4.

43. The dental material as recited in claim 42 wherein m=1 to 4.

44. The dental material according to claim 1 wherein the at least one organohydrogen silicon compound (b) is a QM resin containing Si—H, said QM resin comprising (R⁹)₃SiO_{1/2}, (R⁷)₂(H)SiO_{1/2} and SiO_{4/2} units, wherein R⁷=alkyl, aryl, aralkyl, halogen-substituted alkyl, halogen-substituted alkyl, halogen-substituted aryl, cyanoalkyl, cycloalkyl, or cycloalkenyl and R⁹═R⁷ or H with the proviso that at least 2 Si atoms of the QM resin bear an H atom

45. The dental material according to claim 44 wherein trifunctional (R⁹)₁SiO_{3/2} is present in the QM resin as T units.

46. The dental material according to claim 44 wherein bifunctional R⁷R⁹SiO_{2/2} is present in the QM resin as D units.

47. The dental material according to claim 44 wherein R⁷=methyl, ethyl, isopropyl, phenyl, naphthyl, tolyl, xylyl, benzyl, phenylethyl, 3,3,3-trifluoropropyl, chlorophenyl, or difluorophenyl.

48. The dental material according to claim 44 wherein R⁷=methyl.

49. The dental material according to claim 1 wherein the at least one organohydrogen silicon compound (b) comprises said first structural unit and said second structural unit and has an SiH content of 0.1 to 15 mmol/g, and a viscosity (at 20° C.) of 1 to 10,000 mPa.s.

50. The dental material according to claim 49 wherein said SiH content is 4 to 15 mmol/g and said viscosity (at 20° C.) is 5 to 2,000 mPa.s.

51. The dental material according to claim 49 wherein said SiH content is 7 to 15 mmol/g.

52. The dental material according to claim 1, comprising
(a₁) 1 to 90 wt.-% of at least one compound (a) having at least one rigid or voluminous group and at least two vinyl-functional silyl groups in the molecule, and an Si vinyl content of 0.5 to 10 mmol/g;
(a₂) 0 to 40 wt.-% of at least one organopolysiloxane having at least two vinyl groups in the molecule, and an Si vinyl content of 0.5 to 10 mmol/g and a viscosity of 21 to 350,000 mPa.s,
(b₁) 0 to 90 wt.-% of an organohydrogen silicon compound having at least one rigid or voluminous group and at least two silyl groups having at least two SiH groups, and an SiH content of 0.1 to 15 mmol/g;
(b₂) 0 to 50 wt.-% of an organohydrogen polysiloxane having at least two SiH groups, and an SiH content of 0.1 to 15 mmol/g and a viscosity between 5 and 2,000 mPa.s, wherein the sum of the weight percents of the compounds (b₁) and (b₂) is at least 1 wt.-%;
(c) 0.00001 to 0.2 wt.-% of at least one catalyst for accelerating the hydrosilylation reaction, with reference to pure metal;
(d) 0 to 50 wt.-% of reinforcing fillers having a BET surface of at least 50 m²/g;
(e) 0 to 90 wt.-% of non-reinforcing fillers having a BET surface of less than 50 m²/g and an average grain size of at least 0.1 μm;
(f) 0 to 5 wt.-% of at least one dye;
(g) 0 to 30 wt.-% of at least one moisture binder;
(h) 0 to 1 wt.-% inhibitors;
(i) 0 to 40 wt.-% of at least one QM resin that contains vinyl groups or SiH or SiOR (where R═H or alkyl), having an SiH content of 0 to 15 mmol/g or an SIOR content of 0 to 0.5 mmol/g;
(j) 0 to 80 wt.-% of compounds of organopolysiloxanes that contain vinyl groups, and reinforcing fillers;
(k) 0 to 10 wt.-% of surfactants, emulsifiers or stabilizers;
(l) 0 to 90 wt.-% of radio-opaque substances;
(m) 0 to 20 wt.-% of H₂ absorbers, H₂ adsorbers or substances that reduce or eliminate H₂ development; and
(n) 0 to 20 wt.-% of other aids and additives.

53. The dental material according to claim 52 wherein the dental material comprises:
(a₁) 5 to 75 wt.-% of at least one compound (a) having at least two vinyl-functional silyl groups in the molecule, and an Si vinyl content of 1 to 10 mmol/g;
(a₂) 0 to 30 wt.-% of at least one organo-polysiloxane having at least two vinyl groups in the molecule, and an Si vinyl content of 1 to 10 mmol/g, and a viscosity of 21 to 350,000 mPa.s.
(b₁) 5 to 75 wt.-% of an organohydrogen silicon compound having at least one rigid or voluminous group and at least two silyl groups having at least three SiH groups, and an SiH content of 4 to 15 mmol/g;
(b₂) 0 to 40 wt.-% of an organohydrogen polysiloxane having at least three SiH groups, and an SiH content of 4 to 15 mmol/g, and a viscosity between 5 and 2,000 mPa.s, wherein the sum of the weight percent of the compounds (b₁) and (b₂) is at least 1 wt.-%;
(c) 0.0005 to 0.1 wt.-% of at least one catalyst for accelerating the hydrosilylation reaction, with reference to pure metal;
(d) 0.1 to 40 wt.-% of reinforcing fillers having a BET surface of at least 50 m²/g;
(e) 0 to 80 wt.-% of non-reinforcing fillers having a BET surface of less than 50 m²/g and an average grain size of at least 0.1 μm;
(f) 0 to 2 wt.-% of at least one dye;
(g) 0 to 5 wt.-% of at least one moisture binder;
(h) 0 to 0.6 wt.-% inhibitors;
(i) 0 to 30 wt.-% of at least one QM resin that contains vinyl groups or SiH or SiOR (where R═H or alkyl), having an SiH content of 0 to 1 mmol/g or an SiOR content of 0 to 0.5 mmol/g;
(j) 0 to 50 wt.-% of compounds of organopolysiloxane that contain vinyl groups, and reinforcing fillers;
(k) 0 to 5 wt.-% of surfactants, emulsifiers or stabilizers;
(l) 0 to 80 wt.-% of radio-opaque substances;
(m) 0 to 10 wt.-% of H₂ absorbers, H₂ adsorbers or substances that reduce or eliminate H₂ development; and
(n) 0 to 15 wt.-% of other aids and additives.

54. The dental material according to claim 52 wherein:
said at least one compound (a) is present in an amount of 10 to 60 wt.-% and has an Si vinyl content of 2 to 10 mmol/g;
said at least one organopolysiloxane is present in an amount of 0 to 20 wt.-% and has an Si vinyl content of 2 to 10 mmol/g;
said organohydrogen silicon compound is present in an amount of 10 to 60 wt.-% and has at least three SiH groups and an SiH content of 7 to 15 mmol/g;
said organohydrogen polysiloxane is present in an amount of 0 to 30 wt.-% and has at least three SiH groups and an SiH content of 7 to 15 mmol/g;
said reinforcing fillers are present in an amount of 0.5 to 35 wt.-%;
said non-reinforcing fillers are present in an amount of 0 to 75 wt.-%;
said inhibitors are present in an amount of 0 to 0.1 wt.-%; and
(i) said at least one QM resin is present in an amount of 0 to 20 wt.-%.

55. The dental material according to claim 52 wherein said organohydrogen polysiloxane has an SiH content of 7 to 13 mmol/g.

56. A two-component dental material addition-crosslinking by way of hydrosilylation, comprising:
(a) at least one compound having at least two vinyl groups in the molecule;
(b) at least one organohydrogen silicon compound; and
(c) at least one catalyst;
wherein the at least one compound (a) or the at least one compound (b) comprises a first structural unit and a second structural unit;
said first structural unit comprising at least one voluminous or rigid group;
said second structural unit comprising at least two alkenyl-functional or at least two hydrogen-functional silyl units having the general formula I 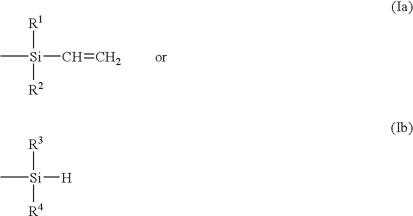
where R¹, R², independent of one another, are selected from the group that consists of alkyl groups, alkenyl groups, aryl groups, aralkyl groups, alkylaryl groups, halogenated alkyl groups, halogenated aryl groups, halogenated aralkyl groups, halogenated alkylaryl groups, cyanoalkyl groups, siloxy groups, cycloalkyl groups, and cycloalkenyl groups, and
R³, R⁴, independent of one another, are H or R¹;
wherein the first and second structural unit are bound
(i) directly;
(ii) by way of an oxygen atom;
(iii) by way of a spacer group; or
(iv) by way of a spacer group bound to the first structural unit by way of an oxygen atom,
and a voluminous or rigid group E or the second structural unit is a linear or cyclic monosiloxane, oligosiloxane, or polysiloxane unit.

57. The dental material according to claim 56 wherein said second structural unit comprises at least three hydrogen-functional silyl units having the general formula I wherein R¹, R², R³ and R⁴ are alkyl groups.

58. The dental material according to claim 56 wherein R¹, R², R³ and R⁴ are methyl groups.

59. A bite impression material for use in dental medicine or dental technology comprising the dental material according to claim 1.

60. A dental cement comprising the dental material according to claim 1.

61. A temporary crown and bridge material for use in dental medicine or dental technology comprising the dental material according to claim 1.

62. A temporary filling material for use in dental medicine or dental technology comprising the dental material according to claim 1.

63. A permanent filling material for use in dental medicine or dental technology comprising the dental material according to claim 1.
